(19) United States
(12) Patent Application Publication
(10) Pub. No.: US 2015/0190421 A1
(43) Pub. Date: Jul. 9, 2015

(54) POLOXAMERS FOR THE IMPROVEMENT OF RESPIRATORY FUNCTION

(71) Applicant: PHRIXUS PHARMACEUTICALS, INC., ANN ARBOR, MI (US)

(72) Inventor: Bruce Edward Markham, Novi, MI (US)

(21) Appl. No.: 14/415,478

(22) PCT Filed: Jul. 18, 2013
(86) PCT No.: PCT/US2013/051101

§ 371 Date: Jan. 16, 2015

Related U.S. Application Data
(60) Provisional application No. 61/673,087, filed on Jul. 18, 2012.

(51) Int. Cl. A61K 31/765; A61K 45/06; A61K 31/573

(52) U.S. Cl. CPC A61K 31/765; A61K 31/573; A61K 45/06

(57) ABSTRACT

Methods for treating or preventing a respiratory deficiency associated with a diaphragm muscle deficiency in a subject are provided, the method comprises administering to the subject in need thereof, a therapeutically effective amount of a poloxamer, for example, poloxamer P-188.

### CROSS REFERENCE TO RELATED APPLICATIONS

This application claims the benefit of U.S. Provisional Patent Application Ser. No. 61/673,087, filed Jul. 18, 2012, the disclosure of which is incorporated herein by reference in its entirety.

### FIELD OF THE INVENTION

The present invention relates to compositions and methods for treating and preventing deficiencies in respiratory function.

### BACKGROUND OF THE INVENTION

Duchenne Muscular Dystrophy (DMD) is an X-linked genetic disease caused by a mutation in the dystrophin gene. As a result, muscles from patients with DMD lack dystrophin, a 427 kDa protein located on the cytoplasmic surface of the plasma membrane, the sarcolemma, of muscle fibers (see, e.g., Blake D J, et al., (2002) Physiol Rev 82, 291-329). Dystrophin is required for the assembly of the dystrophin-associated glycoprotein complex that is embedded in the sarcolemma (see, e.g., Ohlendieck K & Campbell K P (1991) J. Cell. Biol. 115, 1685-1694). The dystrophin-glycoprotein complex links the actin cytoskeleton to the basement membrane and is thought to provide mechanical stability to the sarcolemma (see, e.g., Petrof B J (2002) Am. J. Phys. Med. Rehabil. 81, S162-S174). Although the exact function of dystrophin is still unknown, the pathology demonstrated by the skeletal muscles of young males that lack dystrophin is clear. Boys with DMD experience progressive muscle weakness beginning at about 2-5 years of age, are wheelchair bound by age 12, and die in their mid-twenties from respiratory, or cardiac failure (see, e.g., Hoffman E P, et al., (1987) Cell 51, 919-928).

There exists a need for new compositions and new methods for deficiencies in respiratory function in general, and for preventing and/or correcting the underlying bases of respiratory failure in subjects with deficiencies in respiratory function (e.g., generally as well as in dystrophic subjects).

### SUMMARY OF THE INVENTION

The present invention relates to compositions and methods for treating and preventing respiratory deficiencies. In particular, the present invention provides compositions comprising poloxamers (e.g., poloxamer 188—P-188) and methods of using the same for treating and preventing respiratory deficiencies (e.g., respiratory deficiencies associated with dystrophin-deficient skeletal muscle such as the expiratory muscles, diaphragm, heart failure, or other etiologies).

Experiments conducted during the course of development of embodiments for the present invention demonstrated that poloxamers were effective in reducing respiratory deficiencies, improving lung volume and capacity in subjects treated with poloxamers (e.g., P-188).

Accordingly, in certain embodiments, the present invention provides methods for treating a subject with a respiratory deficiency, the method comprising administering to the subject in need thereof, a composition comprising a poloxamer under conditions such that the respiratory deficiency is improved in the subject. In certain embodiments, the present invention provides methods for preventing or treating respiratory deficiency in a dystrophin deficient subject.

In some embodiments, the present invention relates to a method for the treatment and/or prevention of a respiratory deficiency associated with a deficiency of the expiratory muscles and/or a deficiency of the diaphragm muscle in a subject. In various embodiments, the method comprises administering to the subject in need thereof, a therapeutically effective amount of a poloxamer as described herein, for example, a therapeutically effective dose of poloxamer P-188. In other embodiments, the present invention also relates to methods for increasing the tidal volume, tidal volume/body weight ratio, and decreasing diaphragm muscle fiber degeneration in a subject with a respiratory deficiency, the method comprising, administering to a subject in need of increasing the tidal volume, tidal volume/body weight ratio, and decreasing diaphragm muscle fiber degeneration, a therapeutically effective amount of a poloxamer.

The methods are not limited to a particular type of poloxamer. In some embodiments, the poloxamer is a purified or fractionated or unfractionated poloxamer. In some embodiments, the poloxamer is P-188, P138, P237, P288, P124, P338, and/or P407. In some embodiments, poloxamines and/or polyglycidols are used instead of, or with, poloxamers. The methods are not limited to a particular type of subject. In some embodiments, the subject is a human subject. In some embodiments the subject is a non-human subject (e.g., a mouse). In some embodiments, the subject is a dystrophin deficient subject. In some embodiments, the subject has Duchenne's muscular dystrophy. In some embodiments, the subject additionally suffers from heart failure. The methods are not limited to a particular form of administration of the composition. In some embodiments, the composition is administered via intravenous administration. In some embodiments, the administration is subcutaneous.

In some embodiments, it is expected that each dose of poloxamer comprises an amount of the poloxamer ranging from about 0.01 mg/kg to about 500 mg/kg, or from about 0.01 mg/kg to about 400 mg/kg, or from about 0.01 mg/kg to about 300 mg/kg, or from about 0.01 mg/kg to about 200 mg/kg, or from about 0.01 mg/kg to about 100 mg/kg, or from about 0.01 mg/kg to about 50 mg/kg, or from about 0.01 mg/kg to about 30 mg/kg, or from about 0.01 mg/kg to about 20 mg/kg, or from about 0.01 mg/kg to about 10 mg/kg, or from about 0.01 mg/kg to about 5 mg/kg, or from about 0.01 mg/kg to about 3 mg/kg, or from about 0.01 mg/kg to about 1 mg/kg, or from about 0.01 mg/kg/day to about 0.1 mg/kg weight of a subject is a therapeutically effective dose.

In some embodiments, each dose comprises 3 mg of poloxamer per kg weight of the subject being treated. In some embodiments, each dose comprises 30 mg of poloxamer per kg weight of the subject being treated. In some embodiments, each dose comprises a therapeutically effective dose of poloxamer ranging from 3 mg/kg to about 30 mg/kg.

The methods are not limited to a particular manner of administration of poloxamer to a subject. Examples of administration include, but are not limited to, intravenous, intraarterial, subcutaneous, intraperitoneal, intramuscular injection or infusion, intrathecal and intraventricular administration.

In some embodiments, the composition comprising a poloxamer is co-administered with one or more agents useful in treating skeletal muscle deficiencies. Examples of such agents include, but not limited to, streptomyocin, prednisone, deflazacort, azathioprine, cyclosporine, valproic acid, phenylbutyrate, sodium butyrate, M344, suberoylanilide hydroxamic acid, and PCT124, or a combination of these agents.

In some embodiments, the composition comprising a poloxamer is co-administered with one or more agents useful in treating respiratory deficiencies, including Acute Respiratory Distress Syndrome (ARDS), allergy, asthma, bronchitis, cystic fibrosis, emphysema, lung disease, pneumonia, sinus infections, smoking cessation, and others. Agents useful in combination with the composition comprising a poloxamer include for example, albuterol, beclomethasone, cromolyn sodium, epinephrine, prednisone, flunisolide, ipratropium bromide, isoproterenol sulfate, metaproterenol, nedocromil, oxymetazoline, phenylephrine, pseudoephedrine, salmeterol, terbutaline, and theophylline.

In certain embodiments, the present invention provides compositions comprising a poloxamer and an agent useful for the treatment of a skeletal muscle contraction force deficit. In some embodiments, the agent useful for the treatment of a skeletal muscle contraction force deficit is selected from the group consisting of streptomyocin, prednisone, deflazacort, azathioprine, cyclosporine, valproic acid, phenylbutyrate, sodium butyrate, M344, suberoylanilide hydroxamic acid, and PCT124®, or a combination of these agents. In some embodiments, the composition comprises two or more agents useful for the treatment of a skeletal muscle contraction force deficit.

The compositions are not limited to a particular type of poloxamer. In some embodiments, the poloxamer is P-188, P138, P237, P288, P124, P338, and/or P407. In some embodiments, poloxamines and/or polyglycidols are used instead of, or with, poloxamers.

### BRIEF DESCRIPTION OF THE DRAWINGS

FIG. 1 shows a line graph representing the results of a trial using live wild-type mice and mdx mice after treatment with a FIG. A. 30 mg/kg P-188, FIG. B. 3 mg/kg P-188 and FIG. C. predisone when compared to wild-type mice treated with saline and measuring changes in tidal volume of the tested subjects.

FIG. 2 shows line graphs of mouse diaphragm muscle structural parameters and their change as a result of exposure to saline, poloxamer P-188 and prednisone. FIG. 2A represents the percentage of fibers with centralized nuclei as determined from approximately 600 fibers/animal after treatment with the various compositions; FIG. 2B. represents cross-sectional area of muscle fibers obtained from approximately 50 fibers per animal after treatment with the various compositions; and FIG. 2C represents fiber density as determined from 3 microscopic fields/animal after treatment with the various compositions. N=11 for mdx 3 and N=12 for all other groups. Mean±S.E.M is shown. *P<0.001, ^{#}P=0.01 to 0.001.

FIG. 3 is a line graph measuring the plasma levels of poloxamer P-188 in wild-type after administration at concentrations of 30 mg/kg; 100 mg/kg and 300 mg/kg into the mice.

### DEFINITIONS

The term “wherein said symptoms are reduced” refers to a qualitative or quantitative reduction in detectable symptoms, including but not limited to improved respiratory function, such as measure by any commonly used measure of lung capacity, input or output.

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Background” and “Summary”) and sub-headings used herein are intended only for general organization of topics within the present invention, and are not intended to limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. Any discussion of the content of references cited in the Introduction is intended merely to provide a general summary of assertions made by the authors of the references, and does not constitute an admission as to the accuracy of the content of such references. All references cited in the “Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it is also envisioned that Parameter X may have other ranges of values including 1-9, 2-9, 3-8, 1-8, 1-3, 1-2, 2-10, 2.5-7.8, 2-8, 2-3, 3-10, and 3-9, as mere examples.

Although the open-ended term “comprising,” as a synonym of terms such as including, containing, or having, is use herein to describe and claim the present invention, the invention, or embodiments thereof, may alternatively be described using more limiting terms such as “consisting of” or “consisting essentially of” the recited ingredients.

The terms “respiratory deficiency” or “respiratory insufficiency” relate to a defect and/or insufficiency or inability to adequately ventilate and/or oxygenate, and/or exchange carbon dioxide as a result of defective expiratory muscle activity and/or diaphragm function and/or support, most commonly as a result of a deficiency associated with expiratory and/or diaphragm muscular activity, for example, respiratory deficiencies associated with a dystrophin-deficient expiratory and/or diaphragm muscle. Exemplary respiratory deficiencies can include respiratory failure, hypoxia, anoxia, hypoxemia, hypoxic hypoxia, tachypnea, tachycardia, heart failure, syncope, orthopnea, dyspnea, polycythenemia, cyanosis, chronic obstructive pulmonary disease COPD, asthma, respiratory infections, for example, pneumonia, and bronchitis. Respiratory deficiency as a result of the loss of respiratory muscle strength, with ensuing ineffective cough and decreased ventilation, leads to pneumonia, atelectasis, and respiratory insufficiency in sleep and while awake.

As used herein, the term “at risk for respiratory deficiency” refers to subjects (e.g., a segment of the world population, or research animals) that have an increased risk (i.e. over the average subject (e.g., person or research animal) for a respiratory deficiency (e.g., a subject with DMD) and can occur at any age.

The term “composition” as used herein is intended to encompass a product comprising the specified ingredients (and in the specified amounts, if indicated), as well as any product which results, directly or indirectly, from combination of the specified ingredients in the specified amounts. By “pharmaceutically acceptable” it is meant that the diluent, excipient or carrier must be compatible with the other ingredients of the formulation and not deleterious to the recipient thereof.

As used herein, the term “therapeutic composition comprising a poloxamer” refers to compositions containing a poloxamer (e.g., P-188), or combination of poloxamers, used for the compositions and methods for treating and preventing respiratory deficiencies. A therapeutic composition comprising a poloxamer may also comprise one or more other compounds or agents including, but not limited to, streptomyocin, prednisone, deflazacort, azathioprine, cyclosporine, valproic acid, phenylbutyrate, sodium butyrate, M344, suberoylanilide hydroxamic acid, and PTC124®, or a combination of these agents, and/or other therapeutic agents, physiologically tolerable liquids, gels, carriers, diluents, excipients, salicylates, immunosuppressants, antibiotics, binders, fillers, preservatives, stabilizing agents, emulsifiers, and buffers.

As used herein, the terms “host,” “subject” and “patient” refer to any animal, including but not limited to, human and non-human animals (e.g. rodents), non-human primates, ovines, bovines, ruminants, lagomorphs, porcines, caprines, equines, canines, felines, etc.), that is studied, analyzed, tested, diagnosed or treated (e.g. administered therapeutically or prophylactically a composition comprising a poloxamer of the present invention). The terms “host,” “subject” and “patient” are used interchangeably, unless indicated otherwise herein.

As used herein, the terms “therapeutically effective amount” and “effective amount” when used in reference to a composition comprising a poloxamer of the present invention refer to an amount (e.g., a dosage level) sufficient to effect beneficial or desired results (e.g., that are effective at treating or preventing a respiratory deficiency associated with skeletal muscular defects of the diaphragm). An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment (e.g., compositions of the present invention) to a subject (e.g., a subject or in vivo, in vitro, or ex vivo cells, tissues, and organs).

As used herein, the terms “co-administration” and “co-administering” refer to the administration of at least two agent(s) (e.g., a composition comprising a poloxamer and one or more other agents—e.g., prednisone, streptomyocin) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder or reducing the adverse effects of another drug or medicament resulting from the administration of one or more prophylactic or therapeutic agents or compositions to a patient with such a disease or disorder. In some embodiments, the terms refer to the administration of a poloxamer provided herein, with or without other additional active agent, after the onset of symptoms of the particular disease.

The term “treatment” refers to both therapeutic treatment and prophylactic or preventative measures. For example, those who may benefit from treatment with compositions and methods of the present invention include those already with a disease and/or dysfunction (e.g., respiratory deficiency) as well as those in which a disease and/or dysfunction is to be prevented (e.g., using a prophylactic treatment of the present invention).

As used herein, the term “at risk for disease or dysfunction” refers to a subject (e.g., a human) that is predisposed to experiencing a particular disease or dysfunction. This predisposition may be genetic (e.g., a particular genetic tendency to experience the disease, such as heritable disorders), or due to other factors (e.g., environmental conditions, hypertension, activity level, metabolic syndrome, etc.). Thus, it is not intended that the present invention be limited to any particular risk, nor is it intended that the present invention be limited to any particular type of disorder or dysfunction related to other disorders (e.g., DMD).

As used herein, the term “suffering from disease or dysfunction” refers to a subject (e.g., a human) that is experiencing a particular disease or dysfunction. It is not intended that the present invention be limited to any particular signs or symptoms, nor disease. Thus, it is intended that the present invention encompass subjects that are experiencing any range of disease or dysfunction wherein the subject exhibits at least some of the indicia (e.g., signs and symptoms) associated with the particular disease or dysfunction.

The terms “compound” and “agents” refer to any chemical entity, pharmaceutical, drug, and the like that can be used to treat or prevent a disease, illness, sickness, or disorder of bodily function. Compounds comprise both known and potential therapeutic compounds. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of disease or dysfunction.

### DETAILED DESCRIPTION OF THE INVENTION

Approximately 1 in every 3500 males is affected with Duchenne Muscular Dystrophy (DMD) while Becker Muscular Dystrophy (BMD) is less common affecting approximately 1 in every 30,000 males. Both diseases are the result of mutations in the gene located on the X chromosome, at Xp21.1 that encodes dystrophin. In DMD, dystrophin is absent while in BMD it is either reduced or abnormal in size. Dystrophin is a structural protein that participates in cellular organization in muscle cells and promotes both myofibrillular and sarcolemma (muscle cell membrane) stability (see, e.g., Kaprielian and Severs, 2000 Heart Failure Reviews 5: 221-238).

Discovered in 1984, the mdx mouse (see, e.g., Bulfield G, et al, (1984) Proc. Natl. Acad. Sci. USA 81, 1189-1192) lacks dystrophin and is an important model for studying the effects of dystrophin deficiency. Studies performed on muscle tissue from the mdx mouse have documented impairments in structure and function (see, e.g., Lynch G S, et al., (2001) J Physiol 535, 591-600; Head S I, et al., (1992) Proc. Biol. Sci. 248, 163-169) that include a high degree of susceptibility to contraction-induced force deficits associated with lengthening contractions (see, e.g. Li S, et al, (2006) Hum. Mol. Genet. 15, 1610-1622). Depending on the type of muscle fiber and severity of the injury-producing protocol, dystrophic skeletal muscles sustain a loss in force that is 2 to 7 times greater than muscles of wild-type (WT) mice exposed to the same lengthening contraction protocol (see, e.g, Li S, et al., (2006) Hum. Mol. Genet. 15, 1610-1622). In addition, dystrophic muscles have a sarcolemma that is more permeable to extracellular ions and membrane impermeable dyes (see, e.g., Yeung E W, et al., (2005) J. Physiol. 562, 367-380; Vandebrouck C, et al., (2002) J. Cell Biol. 158, 1089-1096; Petrof B J (2002) Am. J. Phys. Med. Rehabil. 81, S162-S174). Potential pathways responsible for allowing the influx of normally-excluded extracellular constituents into dystrophic muscle fibers include micromembrane tears (see, e.g., Petrof B J (2002) Am. J. Phys. Med. Rehabil. 81, S162-S174) and malfunctioning ion channels (see, e.g., Yeung E W, et al., (2005) J. Physiol. 562, 367-380; Vandebrouck C, et al., (2002) J. Cell Biol. 158, 1089-1096). These pathways allow extracellular calcium to enter muscle fibers and result in an elevated intracellular concentration of calcium. The elevated calcium concentration is deleterious to muscle structure and function through a variety of calcium-dependent mechanisms (see, e.g., Lamb G D, et al., (1995) J. Physiol. 489 (Pt 2), 349-362; Verburg E, et al., (2005) J. Physiol. 564, 775-790). Despite a general acceptance that membranes of dystrophic skeletal muscles are “leakier” than those of WT mice (see, e.g., Yeung E W, et al., (2005) J. Physiol. 562, 367-380; Vandebrouck C, et al., (2002) J. Cell Biol. 158, 1089-1096; Petrof B J (2002) Am. J. Phys. Med. Rehabil. 81, S162-S174), the relative contributions of micromembrane tears and dysfunctional ion channels to the contraction-induced force deficit have not been established.

Streptomycin is an inhibitor of stretch-activated channels (SAC) (see, e.g., Sokabe M, et al., (1993) Ann. N Y Acad. Sci. 707, 417-420) that reduces the magnitude of contraction-induced injury in dystrophic muscles (see, e.g., Yeung E W, et al., (2005) J. Physiol. 562, 367-380). Poloxamer 188 (P-188) is an 8.4 kDa amphiphilic polymer that effectively patches disrupted membranes in neurons (see, e.g., Marks J D, et al., (2001) FASEB J 15, 1107-1109) and cardiac myocytes (see, e.g., Yasuda S, et al., (2005) Nature 436, 1025-1029). P-188 also enhances recovery of skeletal muscle (see, e.g., Lee R C, et al., (1992) Proc. Natl. Acad. Sci. USA 89, 4524-4528), fibroblasts (see, e.g., Merchant F A, et al., (1998) J. Surg. Res. 74, 131-140) and the spinal cord (see, e.g., Borgens R B, et al., (2004) J. Neurosci. Res. 76, 141-154) from a variety of injury-inducing protocols.

Current therapeutic paradigms for DMD are focused on the expression of dystrophin, through exon skipping or viral transduction of truncated dystrophin, or other genes (e.g., utrophin or dysferlin) that limit the consequences of dystrophin deficiency (See, e.g., Gregorevic, et al, Nat Med 10, 828-34 (2004); Squire et al, Hum. Mol. Genet. 11, 3333-44 (2002); Torrente et al., J. Clin. Invest. 114, 182-95 (2004); Goyenvalle et al., Science 306, 1796-9 (2004)). These strategies are promising but are challenging due to the requisite targeting of all striated muscle in the body. The present invention provides a comparatively simple chemical-based alternative for treating DMD comprising administering to a subject with DMD a composition comprising a poloxamer. Although an understanding of the mechanism is not necessary to practice the present invention and the present invention is not limited to any particular mechanism of action, in some embodiments, administration of a poloxamer results in acute membrane stabilization and/or repair, and consequent improvement of respiratory function.

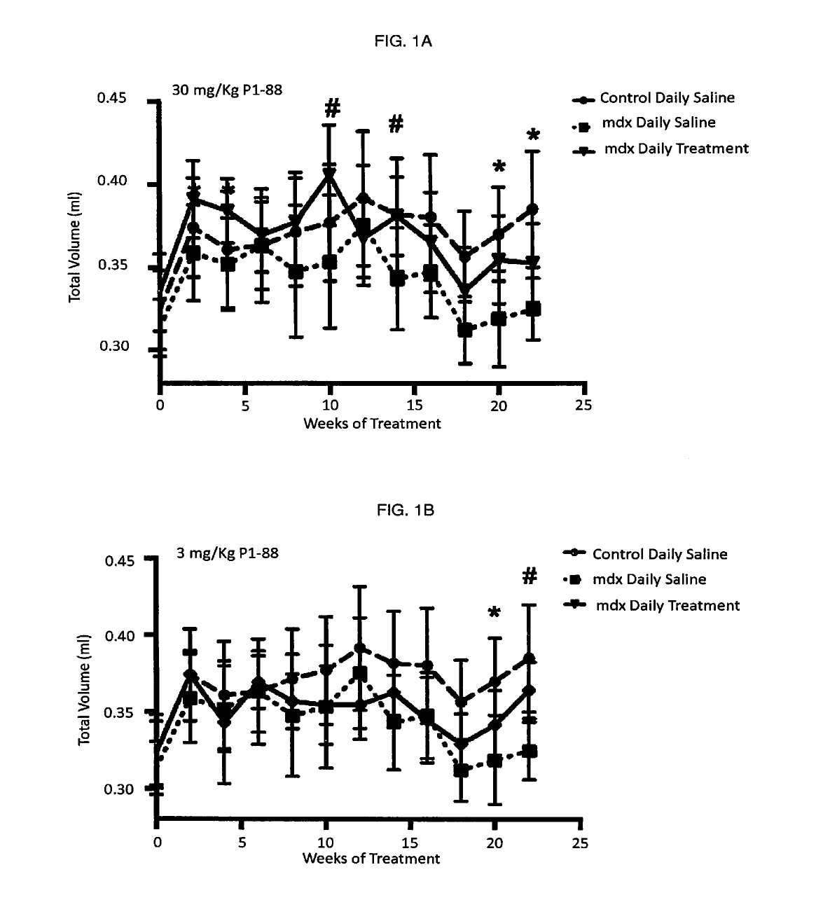

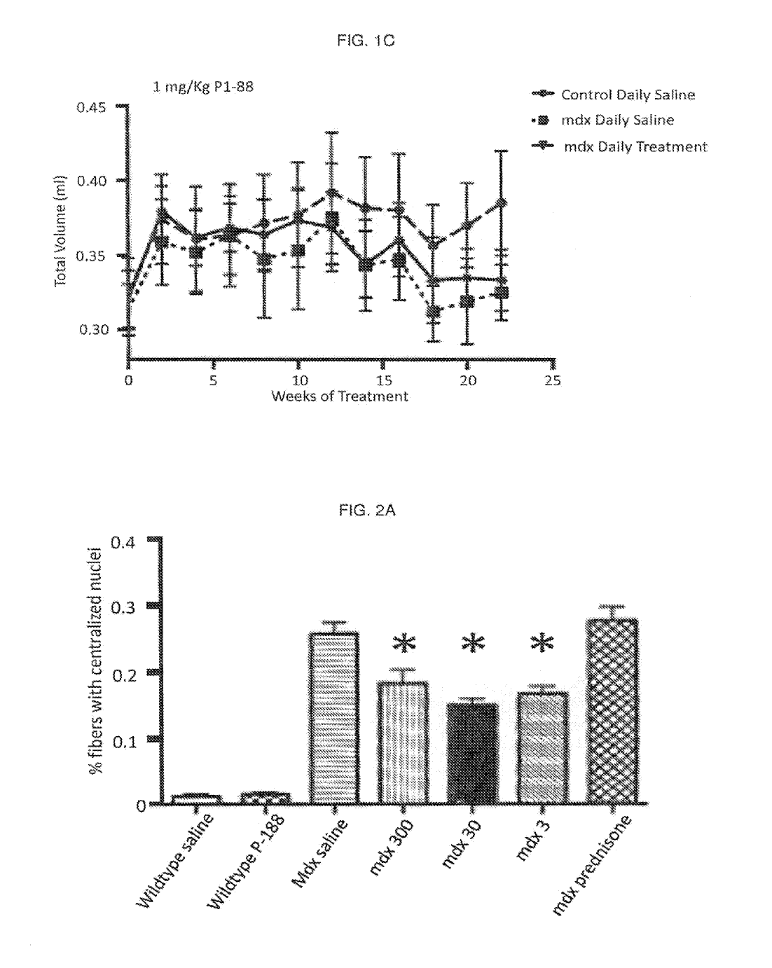

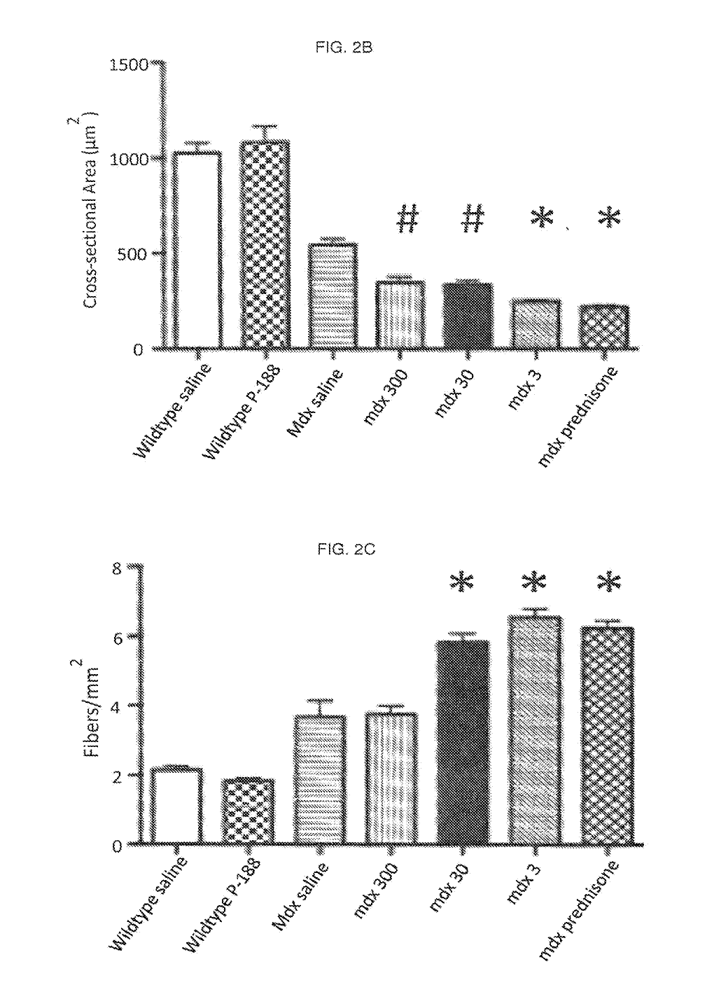

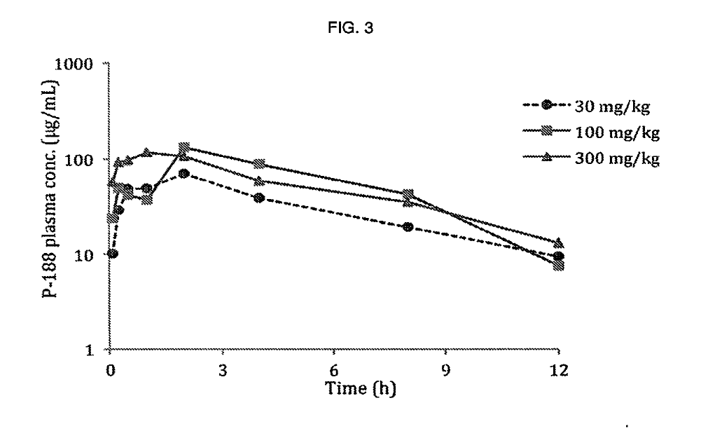

As demonstrated herein (e.g., in mouse models of DMD; See FIGS. 1 and 2), administration of a poloxamer provides ready and immediate improvement for respiratory function. Currently, P 188 is in phase III clinical trials for the treatment of vaso-occlusive crisis in sickle-cell anemia patients, having recently demonstrated the safety and non-toxicity of P-188 in humans (See, e.g., Adams-Graves et al., Blood 90, 2041-6 (1997)). However, unlike the episodic course of sickle-cell anemia, DMD is a progressive disease, and effective poloxamer therapy, in some embodiments, utilizes chronic intravascular administration. Thus, the present invention utilizes membrane sealing poloxamers that represent a new class of therapeutic agents for preventing or limiting progressive damage to DMD, and for treating respiratory dysfunction.

The present invention is not limited to any particular poloxamer. In some preferred embodiments, P188 is used (e.g., in a composition (e.g., pharmaceutical composition) of the present invention). The present invention is not limited to use of P 188. Indeed, any poloxamer that possesses similar characteristics and traits (e.g., biological effects) with those of P 188 find use in the present invention including, but not limited to, P138, P237, P288, P124, P338, and P407.

P-188 is one of a family of poloxamer molecules originally developed by BASF in the 1950s. It is a nonionic triblock co-polymer made of poly(ethylene oxide)₈₀-poly(propylene oxide)₃₀-poly(ethylene oxide)₈₀ (molecular mass˜8.4 Kda). The molecule has several names including PLURONIC F68, RheothRx, and FLOCOR.

Poloxamers (also termed PLURONIC block polymers, available from BASF Corp., Wyandotte, Mich.) generally comprise ethylene oxide (EO) and propylene oxide (PO) blocks arranged in a basic A-B-A structure: EO-PO-EO. This arrangement results in an amphiphilic copolymer, in which the number of hydrophilic EO₍ₓ₎ and hydrophobic PO(y) units can be altered (See, e.g., Reeve, pgs. 231-249, in Handbook of Biodegradable Polymers, Harwood Academic Pub., Eds. Domb et al., (1997)). Copolymers with various x and y values are characterized by distinct hydrophilic-lipophilic balance (HLB). Poloxamers can be synthesized by sequential addition of PO and EO monomers in the presence of an alkaline catalyst, such as sodium or potassium hydroxide (See, e.g., Schmolka, J. Am. Oil Chem. Soc. 54 (1977) 110-116). The reaction is initiated by polymerization of the PO block followed by the growth of EO chains at both ends of the PO block. Anionic polymerization usually produces polymers with a relatively low polydispersity index (M/M).

In some embodiments, a composition or pharmaceutical composition comprising a poloxamer of the present invention comprises a purified and/or fractionated and/or unfractionated poloxamer (e.g., purified and/or fractionated using gel filtration or chromatographic fractionation, or a national formulary unfractionated poloxamer (e.g. USP National Formulary) which is unfractionated (See, e.g., Emanuele et al., Expert Opin. Investig. Drugs. 1998; 7:1193-20, U.S. Pat. Nos. 6,977,045 and 6,761,824). In some embodiments, poloxamers are used that have admixtures (e.g., PO homopolymer and/or block copolymer admixtures) removed. In some embodiments, a poloxamer (e.g., polyoxypropylene/polyoxyethylene copolymer) is used that is optimized for improved biological activity (See, e.g., U.S. Pat. No. 6,747,064). In some embodiments, chemically modified forms of one or more poloxamers are utilized in the compositions and methods of the present invention. Chemical modifications of poloxamers include, but are not limited to, radiolabelling, acetylating, biotinylation, addition of a fluorophore, and other chemical modifications.

A variety of poloxamers can be used in (e.g., in a composition comprising a poloxamer) the present invention that possess similar characteristics and traits (e.g., biological effects) with those of P-188. These poloxamers include, but are not limited to, P138, P237, P288, P124, P338, and P407.

In some embodiments, a poloxamer with a molecular weight of between 5000 and 9000 daltons is used (e.g., in a composition (e.g., pharmaceutical composition) of the present invention). In some embodiments, a poloxamer with a molecular weight of between 9000 and 12000 daltons is used (e.g., in a composition (e.g., pharmaceutical composition) of the present invention). In some embodiments, a poloxamer with a molecular weight of between 12000 and 15000 daltons is used. A poloxamer with a molecular weight below 5000 or greater than 15000 daltons may also find use in the present invention (e.g., in a composition (e.g., pharmaceutical composition) of the present invention).

In some embodiments, a poloxamer with a polyoxy ethylene content greater than 50% is used (e.g., in a composition (e.g., pharmaceutical composition) of the present invention). In some embodiments, a poloxamer with a polyoxy ethylene content between 50 and 60% is used. In some embodiments, a poloxamer with a polyoxyethylene content between 60 and 70% is used. Poloxamers with a polyoxyethylene content below 50% and above 70% may also find use in the present invention (e.g., in a composition (e.g., pharmaceutical composition) of the present invention).

Some common biological uses of P-188 include use as a stool softener in several commercially available laxatives, as an ingredient in cosmetics and as an emulsifier for pharmaceutical agents. It is a powerful surfactant. P 188 has been shown to insert into lipid monolayers (See, e.g., Maskarinec et al., 2002 Biophys. J. 82: 1453-1459). It has many biological effects in vivo including the repair of electrically damaged cell membranes (See, e.g., Lee et al., (1992) Proc. Natl. Acad. Sci. USA 89: 4524-4528), in controlled drug delivery, for sensitizing tumors to chemotherapy (See, e.g., Kabanov et al., Adv. Drug Deliv. Rev. 2002, 54, 759-779), and for delivery of gene therapies, among others. Additionally, P 188 was shown to have an effect on blood flow and viscosity as well as platelet adhesiveness. (See, e.g., Graver et al., (1969) Circ. 39 and 40: 1249, (Suppl. I)). It was developed as a therapeutic agent under the name of RheothRx by Glaxo Welcome (See, e.g., Adams-Graves et al., (1997), Blood 90: 2041-2046) and by CytRx under the name of FLOCOR for vaso-occlusive crisis in sickle cell disease and has been in phase III clinical trials (See, e.g., Emanuele, (1998) Expert Opin. Investig. Drugs 7:1193-1200). It was also in Phase III trials to assess thrombolytic activity in patients with acute myocardial infarction (MI) (CORE), with mixed results (Schaer et al., (1996) Circ. 94: 298-307; Chareonthaitawe et al., (2000) Heart 84: 142-148). It has been in Phase II trials as an adjunct to primary percutaneous transluminal coronary angioplasty for acute MI (See, e.g., O'Keefe, et al., 1996 Am. J. Cardiol. 78: 747-750). Thus, the present invention contemplates use of poloxamers (e.g., P138, P237 and P288) that enjoy similar characteristics and biological effects to those of P 188 (e.g., to treat and/or prevent skeletal muscle deficiencies (e.g., dystrophin-deficient skeletal muscle; skeletal muscle having a contraction force deficit; skeletal muscle having a Ca²⁺ imbalance; skeletal muscle having microtears).

P 188 is safe when given acutely for up to 72 hr (See, e.g., Adams-Graves et al., (1997), Blood 90: 2041-2046) and is well tolerated in children and adults upon repeated exposure (See, e.g., Gibbs and Hagemann, 2004 Ann. Pharmacother. 38: 320-324). The most significant adverse effect in studies with RheothRx was renal dysfunction but this was not seen with the more highly purified form FLOCOR. The most frequently experienced adverse effects were pain, injection site abnormalities and nausea. It has a half-life in plasma of 7.5 hr in rodents and 18 hr in human subjects. Pharmacokinetic studies have shown that <5% of purified poloxamer is metabolized. A single metabolite of higher molecular weight and slower clearance has been detected (See, Gibbs and Hagemann, 2004 Ann. Pharmacother. 38: 320-324). Renal clearance is the primary route of elimination.

In some embodiments, a composition comprising a poloxamer (e.g., P-188) provides additive or synergistic benefits when administered with one or more compositions (e.g, pharmaceuticals, drugs, etc.) (e.g., streptomycin) used currently for treating DMD (e.g., prednisone, deflazacort, azathioprine, cyclosporine, valproic acid, phenylbutyrate, sodium butyrate, M344, suberoylanilide hydroxamic acid, and PTC124® (Ataluren)).

The present invention is not limited by the type of subject administered a composition of the present invention. Indeed, a wide variety of subjects are contemplated to be benefited from administration of a composition of the present invention. In preferred embodiments, the subject is a human. In some embodiments, human subjects are of any age (e.g., adults, children, infants, etc.) that have or are likely to suffer from a respiratory deficiency associated with a diaphragm muscle deficiency (e.g., a subject with DMD). In some embodiments, the subjects are non-human mammals (e.g., pigs, cattle, goats, horses, sheep, or other livestock; or mice, rats, rabbits or other animal commonly used in research settings).

The present invention further provides pharmaceutical compositions (e.g., comprising a poloxamer described herein). A composition comprising a poloxamer of the present invention can be used or as a prophylactic. A composition comprising a poloxamer of the present invention can be administered to a subject via a number of different delivery routes and methods.

In preferred embodiments, a composition comprising a poloxamer of the present invention is administered via subcutaneous administration; or is administered via intravenous (IV) administration. In some embodiments, a composition of the present invention may be administered one or more times a day for several days. In some embodiments, a composition of the present invention may be administered one or more times a day for more than one week. In some embodiments, a composition of the present invention may be administered one or more times a day for two or more weeks. In some embodiments, a composition of the present invention may be administered one or more times a day for one or more months, two or more months, four or more months, eight or more months, or for more than a year. In preferred embodiments, a composition of the present invention is administered (e.g., via chronic administration (e.g., administered one, two, three or more times a week in a physician's office for a duration (e.g., over a period of weeks, months or years) that is sufficient to improve skeletal muscle contraction (e.g., by lowering and maintaining skeletal muscle calcium levels at normal levels). The present invention is not limited by intravenous administration. Indeed, any method of administration that introduces a composition of the present invention into the vasculature is contemplated to be useful as a delivery means. For example, in some embodiments, a composition of the present invention is administered via parenteral administration. Examples of parenteral administration include intravenous, intraarterial, subcutaneous, intraperitoneal, intramuscular injection or infusion, intrathecal or intraventricular administration.

Compositions and formulations for parenteral, IV, or other route of administration may include sterile aqueous solutions that may also contain buffers, diluents and other suitable additives such as, but not limited to, penetration enhancers, carrier compounds and other pharmaceutically acceptable carriers or excipients.

A composition of the present invention may be formulated for administration by any route, such as subcutaneous or intravenous, or other route described herein. In some embodiments, a composition of the present invention may comprise sterile aqueous preparations. Acceptable vehicles and solvents include, but are not limited to, water, Ringer's solution, phosphate buffered saline and isotonic sodium chloride solution. In addition, sterile, fixed oils are conventionally employed as a solvent or suspending medium. For this purpose any bland fixed mineral or non-mineral oil may be employed including synthetic mono-ordi-glycerides. In addition, fatty acids such as oleic acid find use in the preparation of injectables. Carrier formulations suitable for IV, parenteral, mucosal, subcutaneous, intramuscular, intraperitoneal, intravenous, oral (e.g., via ingestion) or administration via other routes may be found in Remington: The Science and Practice of Pharmacy, Mack Publishing Company, Easton, Pa., 19th edition, 1995.

In additional preferred embodiments, a composition of the present invention is administered in an amount (e.g., a dose) that is sufficient to improve skeletal muscle contraction (e.g., by lowering and maintaining skeletal muscle calcium levels at normal levels). The present invention is not limited to any particular dose. Indeed, the desired dose may vary depending upon the subject being treated (e.g., the age, health status, and type and/or degree of skeletal muscle deficiency being treated).

A “dose amount” or “dose” as used herein, is generally equal to the dosage of the active ingredient which may be administered once per day, or once per week, or may be administered several times a day (e.g. the unit dose is a fraction of the desired daily dose), or may be administered several times per week (as a fraction of the desired weekly dose) or one or more times per month. For example, an illustrative therapeutically effective dose amount of 1,000 mg/day of the poloxamer may be administered to a subject having a respiratory deficiency as 1 dose of 1,000 mg, 2 doses of 500 mg each or 4 doses of 250 mg each, or 5 doses of 200 mg each per day. The term “unit dose” as used herein may be taken to indicate a discrete amount of the therapeutic composition which comprises a predetermined amount of the active agent. The amount of the poloxamer is generally equal to the dosage of the poloxamer which may be administered once per day, or may be administered several times a day (e.g. the unit dose is a fraction of the desired daily dose), or once per week, or administered several times per week, or administered one or more times per month. The unit dose may also be taken to indicate the total daily dose, which may be administered once per day or may be administered as a convenient fraction of such a dose (e.g. the unit dose is the total daily dose which may be given in fractional increments, such as, for example, one-half or one-third or one-quarter or one-fifth the dosage).

As used herein, the term “daily dose amount” refers to the amount of a poloxamer per day that is administered or prescribed to a patient. This amount can be administered in multiple unit doses or in a single unit dose, in a single time during the day or at multiple times during the day. As used herein, the term “weekly dose amount” refers to the amount of a poloxamer that is administered or prescribed to a patient per week. This amount can be administered in multiple unit doses or in a single unit dose, at a single time during the week or at multiple times during the week.

A pharmaceutical composition of the present invention can be formulated to be compatible with its intended route of administration, as determined by those of skill in the art, and optionally, formulated under FDA-approved methods. Exemplary routes of administration of the pharmaceutical compositions, compositions and formulations can include: parenteral, e.g., subcutaneous, transmucosal, transdermal, intravenous, intramuscular, and intraperitoneal administration. The parenteral preparation can be enclosed in ampoules, disposable syringes or multiple dose, unit dose, or multiple unit dose vials or containers made of glass or plastic.

In some embodiments, the amount of the poloxamer present in the composition, or pharmaceutical composition may be from about 0.01 mg/kg to about 500 mg/kg, or from about 0.01 mg/kg to about 400 mg/kg, or from about 0.01 mg/kg to about 300 mg/kg, or from about 0.01 mg/kg to about 200 mg/kg, or from about 0.01 mg/kg to about 100 mg/kg, or from about 0.01 mg/kg to about 50 mg/kg, or from about 0.01 mg/kg to about 30 mg/kg, or from about 0.01 mg/kg to about 20 mg/kg, or from about 0.01 mg/kg to about 10 mg/kg, or from about 0.01 mg/kg to about 5 mg/kg, or from about 0.01 mg/kg to about 3 mg/kg, or from about 0.01 mg/kg to about 1 mg/kg, or from about 0.01 mg/kg to about 0.1 mg/kg. In some embodiments, the amount of the poloxamer present in the composition, or pharmaceutical composition provides a dose of about 0.01 mg/kg to about 90 mg/kg, or from about 0.01 mg/kg to about 70 mg/kg, or from about 0.01 mg/kg to about 50 mg/kg, or from about 0.01 mg/kg to about 30 mg/kg, or from about 0.01 mg/kg to about 20 mg/kg, or from about 0.01 mg/kg to about 10 mg/kg, or from about 0.01 mg/kg to about 5 mg/kg, or from about 0.01 mg/kg to about 1 mg/kg, or from about 0.01 mg/kg to about 0.1 mg/kg. In several embodiments, the dose may be administered as daily doses and/or weekly doses. In the above exemplary methods, the composition or pharmaceutical composition can be administered to a subject in need thereof at least once per day, for example, at least once per day, or at least twice per day, or every 4 hours, or every 8 hours, or every 12 hours, or every 24 hours, or every 36 hours, or every 48 hours, or one to five times per week, e.g. once per week, or once per two weeks, or once per month, or combinations thereof, for a period of 1-52 weeks, or 1-10 weeks, or at frequencies and/or amounts that improves overall therapy, reduces, avoids or eliminates symptoms, reduces side-effects or causes of a disease or disorder, or enhances the therapeutic efficacy of another therapeutic agent.

In some embodiments, it is expected that each dose comprises a therapeutically effective amount of a poloxamer ranging approximately between 0.01 mg to 500 mg per kg weight of said subject; approximately between 0.01 mg to 400 mg per kg weight of said subject; approximately between 0.01 mg to 300 mg per kg weight of said subject; approximately between 0.01 mg to 200 mg per kg weight of said subject; approximately between 0.01 mg to 100 mg per kg weight of said subject; approximately between 0.01 mg to 50 mg per kg weight of said subject; approximately between 0.01 mg to 30 mg per kg weight of said subject; approximately between 0.01 mg to 20 mg per kg weight of said subject; approximately between 0.01 mg to 10 mg per kg weight of said subject; approximately between 0.1 mg to 500 mg per kg weight of said subject; approximately between 0.1 mg to 400 mg per kg weight of said subject; approximately between 0.1 mg to 300 mg per kg weight of said subject; approximately between 0.1 mg to 200 mg per kg weight of said subject; approximately between 0.1 mg to 100 mg per kg weight of said subject; approximately between 0.1 mg to 50 mg per kg weight of said subject; approximately between 0.1 mg to 30 mg per kg weight of said subject; approximately between 1 mg to 50 mg per kg weight of said subject; approximately between 1 mg to 10 mg per kg weight of said subject; approximately between 3 mg to 30 mg per kg weight of said subject. In the above exemplary methods, the doses comprising a composition or pharmaceutical composition can be administered to a subject in need thereof at least once per day, for example, at least once per day, or at least twice per day, or every 4 hours, or every 8 hours, or every 12 hours, or every 24 hours, or every 36 hours, or every 48 hours, or one to five times per week, e.g. once per week, or once per two weeks, or once per month, or combinations thereof, for a period of 1-52 weeks, or 1-10 weeks, or at frequencies and/or amounts that improves overall therapy, reduces, avoids or eliminates symptoms, reduces side-effects or causes of a disease or disorder, or enhances the therapeutic efficacy of another therapeutic agent. An optimal amount for a particular administration can be ascertained by standard studies involving analysis of diaphragm muscle contraction and diaphragm muscle physiology, as described herein in the examples section, and assays and protocols known in the art.

In some embodiments, a pharmaceutical composition is provided wherein the pharmaceutical composition is used for the prevention and/or treatment of a respiratory deficiency associated with a diaphragm muscle deficiency. In various embodiments, the pharmaceutical compositions of the present invention comprises a therapeutically effective amount of a poloxamer in admixture with at least one pharmaceutically acceptable excipient, and the amount of poloxamer in the pharmaceutically composition ranges from between 0.01 mg to 500 mg per kg weight of said subject; approximately between 0.01 mg to 400 mg per kg weight of said subject; approximately between 0.01 mg to 300 mg per kg weight of said subject; approximately between 0.01 mg to 200 mg per kg weight of said subject; approximately between 0.01 mg to 100 mg per kg weight of said subject; approximately between 0.01 mg to 50 mg per kg weight of said subject; approximately between 0.01 mg to 30 mg per kg weight of said subject; approximately between 0.01 mg to 20 mg per kg weight of said subject; approximately between 0.01 mg to 10 mg per kg weight of said subject; approximately between 0.1 mg to 500 mg per kg weight of said subject; approximately between 0.1 mg to 400 mg per kg weight of said subject; approximately between 0.1 mg to 300 mg per kg weight of said subject; approximately between 0.1 mg to 200 mg per kg weight of said subject; approximately between 0.1 mg to 100 mg per kg weight of said subject; approximately between 0.1 mg to 50 mg per kg weight of said subject; approximately between 0.1 mg to 30 mg per kg weight of said subject; approximately between 1 mg to 50 mg per kg weight of said subject; approximately between 1 mg to 10 mg per kg weight of said subject; approximately between 3 mg to 30 mg per kg weight of said subject. In the above exemplary methods, the pharmaceutical composition can be administered to a subject in need thereof at least once per day, for example, at least once per day, or at least twice per day, or every 4 hours, or every 8 hours, or every 12 hours, or every 24 hours, or every 36 hours, or every 48 hours, or one to five times per week, e.g. once per week, or once per two weeks, or once per month, or combinations thereof, for a period of 1-52 weeks, or 1-10 weeks, or at frequencies and/or amounts that improves overall therapy, reduces, avoids or eliminates symptoms, reduces side-effects or causes of a disease or disorder, or enhances the therapeutic efficacy of another therapeutic agent. An optimal amount for a particular administration can be ascertained by standard studies involving analysis of diaphragm muscle contraction and diaphragm muscle physiology, as described herein in the examples section, and assays and protocols known in the art.

In some embodiments, the pharmaceutical compositions of the present invention comprises a therapeutically effective amount of a poloxamer in admixture with at least one pharmaceutically acceptable excipient, and the amount of poloxamer in the pharmaceutically composition ranges from between about 5 mg to 45,000 mg, or from about 50 mg to about 40,000 mg, or from about 50 mg to about 30,000 mg, or from about 50 mg to about 20,000 mg, or from about 50 mg to about 10,000 mg, or from about 50 mg to about 5,000 mg, or from about 50 mg to about 4,000 mg, or from about 50 mg to about 3,000 mg, or from about 50 mg to about 2,000 mg, or from about 50 mg to about 1,500 mg, or from about 50 mg to about 1,000 mg. The pharmaceutical composition may administered one or more timers per day (for example 1-5 times per day), or one or more times per week (for example, 1-5 times per week), to a subject in need thereof. In the above exemplary methods, the pharmaceutical composition can be administered to a subject in need thereof at least once per day, for example, at least once per day, or at least twice per day, or every 4 hours, or every 8 hours, or every 12 hours, or every 24 hours, or every 36 hours, or every 48 hours, or one to five times per week, e.g. once per week, or once per two weeks, or once per month, or combinations thereof, for a period of 1-52 weeks, or 1-10 weeks, or at frequencies and/or amounts that improves overall therapy, reduces, avoids or eliminates symptoms, reduces side-effects or causes of a disease or disorder, or enhances the therapeutic efficacy of another therapeutic agent.

The above recited exemplary daily doses may be administered as a single daily dose, or may be divided into several doses administered throughout the day, for example, 1 to 5 doses, or two or three doses per day. In some embodiments, the daily dose amount of the poloxamer per day that is administered or prescribed to a patient may be from about 0.01 mg/kg/day to about 100 mg/kg/day, from about 0.05 mg/kg/day to about 100 mg/kg/day, from about 0.1 mg/kg/day to about 100 mg/kg/day, from about 0.5 mg/kg/day to about 100 mg/kg/day, from about 1 mg/kg/day to about 100 mg/kg/day, or from about 1 mg/kg/day to about 50 mg/kg/day, or from about 1 mg/kg/day to about 10 mg/kg/day. In some embodiments, the daily dosed amount of a poloxamer in a composition or pharmaceutical composition may be from about 0.01 mg/kg/day to about 500 mg/kg/day, or from about 0.01 mg/kg/day to about 400 mg/kg/day, or from about 0.01 mg/kg/day to about 300 mg/kg/day, or from about 0.01 mg/kg/day to about 200 mg/kg/day, or from about 0.01 mg/kg/day to about 100 mg/kg/day, or from about 0.01 mg/kg/day to about 50 mg/kg/day, or from about 0.01 mg/kg/day to about 30 mg/kg/day, or from about 0.01 mg/kg/day to about 20 mg/kg/day, or from about 0.01 mg/kg/day to about 10 mg/kg/day, or from about 0.01 mg/kg/day to about 5 mg/kg/day, or from about 0.01 mg/kg/day to about 3 mg/kg/day, or from about 0.01 mg/kg/day to about 1 mg/kg/day, or from about 0.01 mg/kg/day to about 0.1 mg/kg/day. In the above exemplary methods, the composition or pharmaceutical composition can be administered to a subject in need thereof at least once per day, for example, at least once per day, or at least twice per day, or every 4 hours, or every 8 hours, or every 12 hours, or every 24 hours, or every 36 hours, or every 48 hours, or one to five times per week, e.g. once per week, or once per two weeks, or once per month, or combinations thereof, for a period of 1-52 weeks, or 1-10 weeks, or at frequencies and/or amounts that improves overall therapy, reduces, avoids or eliminates symptoms, reduces side-effects or causes of a disease or disorder, or enhances the therapeutic efficacy of another therapeutic agent.

The above recited exemplary weekly doses may be administered as a single weekly dose, or may be divided into several doses administered throughout the week, for example, 1 to 5 doses, or two or three doses per week. In some embodiments, the weekly dose amount of the poloxamer per day that is administered or prescribed to a patient may be from about 0.01 mg/kg/week to about 100 mg/kg/week, from about 0.05 mg/kg/week to about 100 mg/kg/week, from about 0.1 mg/kg/week to about 100 mg/kg/week, from about 0.5 mg/kg/week to about 100 mg/kg/week, from about 1 mg/kg/week to about 100 mg/kg/week, or from about 1 mg/kg/week to about 50 mg/kg/week, or from about 1 mg/kg/week to about 10 mg/kg/week. In some embodiments, the daily dosed amount of a poloxamer in a composition or pharmaceutical composition may be from about 0.01 mg/kg/week to about 500 mg/kg/week, or from about 0.01 mg/kg/week to about 400 mg/kg/week, or from about 0.01 mg/kg/week to about 300 mg/kg/week, or from about 0.01 mg/kg/week to about 200 mg/kg/week, or from about 0.01 mg/kg/week to about 100 mg/kg/week, or from about 0.01 mg/kg/week to about 50 mg/kg/week, or from about 0.01 mg/kg/week to about 30 mg/kg/week, or from about 0.01 mg/kg/week to about 20 mg/kg/week, or from about 0.01 mg/kg/week to about 10 mg/kg/week, or from about 0.01 mg/kg/week to about 5 mg/kg/week, or from about 0.01 mg/kg/week to about 3 mg/kg/week, or from about 0.01 mg/kg/week to about 1 mg/kg/week, or from about 0.01 mg/kg/week to about 0.1 mg/kg/week. In the above exemplary methods, the composition or pharmaceutical composition can be administered to a subject in need thereof in doses at least once per day, for example, at least once per day, or at least twice per day, or every 4 hours, or every 8 hours, or every 12 hours, or every 24 hours, or every 36 hours, or every 48 hours, or one to five times per week, e.g. once per week, or once per two weeks, or once per month, or combinations thereof, for a period of 1-52 weeks, or 1-10 weeks, or at frequencies and/or amounts that improves overall therapy, reduces, avoids or eliminates symptoms, reduces side-effects or causes of a disease or disorder, or enhances the therapeutic efficacy of another therapeutic agent.

In some embodiments, a therapeutically effective amount or a daily and/or weekly dose amount of a poloxamer in a pharmaceutical composition may be from about 0.1 mg/kg to about 300 mg/kg. In some embodiments, the amount of a poloxamer in a pharmaceutical composition may be from about 0.1 mg/kg to about 30 mg/kg. In some embodiments, the amount of a poloxamer in a pharmaceutical composition may be from about 0.1 mg/kg to about 3 mg/kg. In some embodiments, a therapeutically effective amount or a daily dose and/or weekly dose amount of a poloxamer in a pharmaceutical composition may be from about 5 mg to 45,000 mg, or from about 5 mg to about 40,000 mg, or from about 5 mg to about 30,000 mg, or from about 5 mg to about 20,000 mg, or from about 5 mg to about 10,000 mg, or from about 5 mg to about 5,000 mg, or from about 5 mg to about 4,000 mg, or from about 5 mg to about 3,000 mg, or from about 5 mg to about 2,000 mg, or from about 5 mg to about 1,500 mg, or from about 5 mg to about 1,000 mg, the pharmaceutical composition may administered per day, or per week, to a subject in need thereof. In the above exemplary methods, the therapeutically effective amounts or daily and/or weekly doses can be administered to a subject in need thereof at least once per day, for example, at least once per day, or at least twice per day, or every 4 hours, or every 8 hours, or every 12 hours, or every 24 hours, or every 36 hours, or every 48 hours, or one to five times per week, e.g. once per week, or once per two weeks, or once per month, or combinations thereof, for a period of 1-52 weeks, or 1-10 weeks, or at frequencies and/or amounts that improves overall therapy, reduces, avoids or eliminates symptoms, reduces side-effects or causes of a disease or disorder, or enhances the therapeutic efficacy of another therapeutic agent.

In some embodiments, pharmaceutical preparations comprising a poloxamer are formulated in dosage unit form for ease of administration and uniformity of dosage. Dosage unit form, as used herein, refers to a physically discrete unit of the pharmaceutical preparation appropriate for the subject undergoing treatment (e.g., administration of a composition of the present invention). Each dosage should contain a quantity of the compositions comprising a poloxamer calculated to produce the desired response (e.g., improved respiratory function). Procedures for determining the appropriate dosage unit, in addition to being described herein, are well known to those skilled in the art.

Dosage units may be proportionately increased or decreased based on several factors including, but not limited to, the weight, age, concomitant medicaments used, and health status of the subject. In addition, dosage units may be increased or decreased based on the response of the subject to the treatment (e.g., amount of improvement in respiratory function).

In certain embodiments of the invention, compositions or pharmaceutical compositions comprise a therapeutically effective amount of a poloxamer and at least one pharmaceutically acceptable excipient. In various examples, a pharmaceutically acceptable excipient, for example a liquid excipient, for example a solubulizer, a suspending agent, an isotonic agent, a buffering agent or a pH adjusting agent, or combinations thereof. In some embodiments, the pharmaceutical composition is suitable for parenteral administration, for example, a therapeutically effective amount of a poloxamer in admixture with a liquid excipient useful in parenteral administration, e.g. intravenous, intradermal, intraperitoneal, intramuscular, subcutaneous or combinations thereof. In some embodiments, the pharmaceutical composition may comprise one or more alcohols, zinc-containing compounds, emollients, humectants, thickening and/or gelling agents, neutralizing agents, and surfactants. Water used in the formulations is preferably deionized water having a neutral pH. In some embodiments, illustrative liquid excipients can include one or more solutions or suspensions used for intravenous, intradermal, intramuscular, intraperitoneal, or subcutaneous application, which can include one or more of the following components: a sterile diluent such as sterile grade water for injection, physiological saline solution (e.g., phosphate buffered saline (PBS)); antibacterial and antifungal agents such as parabens, chlorobutanol, phenol, ascorbic acid, thimerosal, and the like; antioxidants such as ascorbic acid or sodium bisulfite; chelating agents such as ethylenediaminetetraacetic acid (EDTA); buffers such as acetates, citrates or phosphates and agents for the adjustment of pH, or tonicity such as sodium chloride or dextrose. In many cases, it will be preferable to include isotonic agents, for example, sugars, polyalcohols such as mannitol or sorbitol, and sodium chloride in the composition.

The compositions of the present invention, whether pharmaceutical or not, may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, preservatives, antioxidants, opacifiers, thickening agents and stabilizers. However, such materials, when added, preferably do not unduly interfere with the biological activities of the components of the compositions of the present invention. The formulations can be sterilized and, if desired, mixed with auxiliary agents (e.g., lubricants, preservatives, stabilizers, wetting agents, emulsifiers, salts for influencing osmotic pressure, buffers, colorings, flavorings and/or aromatic substances and the like) that do not deleteriously interact with the poloxamer of the formulation.

The present invention also includes methods involving co-administration of a composition comprising a poloxamer with one or more additional active agents. Indeed, it is a further aspect of this invention to provide methods for enhancing prior art treatment methods and/or pharmaceutical compositions by co-administering a composition of the present invention. In co-administration procedures, the agents may be administered concurrently or sequentially. In one embodiment, the compositions described herein are administered prior to the other active agent(s). The pharmaceutical formulations and modes of administration may be any of those described herein. In addition, the two or more co-administered agents may each be administered using different modes (e.g., routes) or different formulations. The additional agents to be co-administered can be any of the well-known agents in the art, including, but not limited to, those that are currently in clinical use. As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Co-administration encompasses administration of the first and second amounts of the active agents (i.e. a poloxamer, and an additional active agent) in an essentially simultaneous manner, such as in a single pharmaceutical composition, for example, a solution having a fixed ratio of first and second amounts, or in multiple, separate capsules solutions for injections for each. In some embodiments, co-administration encompasses administration of the first and second amounts of the active agents ((i.e. a poloxamer, and an additional active agent) in an essentially simultaneous manner, such as in a single pharmaceutical composition, for example, a capsule, tablet and/or an injectable solution having a fixed ratio of first and second amounts, or in multiple, separate capsules solutions for injections or tablets for one of the active agents. In addition, such co-administration also encompasses use of a first active agent (i.e. a poloxamer), and an additional active agent in a sequential manner in either order. When co-administration involves the separate administration of the first amount of a poloxamer and a second amount of an additional therapeutic agent, the active agents are administered sufficiently close in time to have the desired therapeutic effect. In some embodiments, when co-administration involves the separate administration of the poloxamer and a second amount of an additional therapeutic agent, the active agents are administered sufficiently close in time to have the desired therapeutic effect. For example, the period of time between each administration that can result in the desired therapeutic effect, can range from minutes to hours to days and can be determined taking into account the properties of each active agent such as potency, solubility, bioavailability, plasma half-life and kinetic profile, which may be known individually, or at least ascertainable with respect to the second active agent without undue experimentation. For example, a mixture of a poloxamer, (e.g, P-188) described herein and a second therapeutic agent can be administered in any order within about 24 hours of each other, within about 16 hours of each other, within about 8 hours of each other, within about 4 hours of each other, within about 1 hour of each other or within about 30 minutes of each other. In other embodiments, a poloxamer (e.g, P-188), described herein and a second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a therapy comprising a poloxamer can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second active agent) to a subject. In still other embodiments, a first therapy (e.g., a prophylactic or therapeutic agent such as a therapy comprising a poloxamer, can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second active agent) to a subject.

It is contemplated that the administration of a composition comprising a poloxamer may be co-administered with one or more known therapeutic agents for treating diaphragm muscle deficiencies. For example, agents that are known in the art for treating skeletal muscle deficiencies (e.g., dystrophin-deficient skeletal muscle; skeletal muscle having a contraction force deficit; skeletal muscle having a Ca²⁺ imbalance; skeletal muscle having microtears) include, but are not limited to, streptomyocin, corticosteroids (e.g., prednisone, deflazacort), immunosupressive agents (e.g., azathioprine, cyclosporine), valproic acid, phenylbutyrate, sodium butyrate. M344 (a benzamide and histone deacetylase [HDAC] inhibitor), suberoylanilide hydroxamic acid (SAHA), and PTC124® (Ataluren) (PTC Therapeutics, Inc, South Plainfield, N.J.). PTC124® is an oxadiazole compound that, when taken orally, can override nonsense stop translation signals induced by the dystrophin gene mutation such that the protein produced is the full-length protein (see, e.g., Hamed S A (2006) Drugs November; 9(11): 783-9) (PTC124® is currently in phase 11 clinical trials for patients with Duchenne MD and cystic fibrosis).

In view of the multiple benefits associated with poloxamer compositions, the present invention, pharmaceutical compositions and formulations, whether parenteral or otherwise (e.g. intravenous, intradermal, intraperitoneal, intramuscular, or subcutaneous administration) can be administered to a patient in need of preventative, therapeutic and curative treatment against respiratory deficiencies associated with dysfunctional expiratory muscle and/or diaphragm muscle, for example, respiratory deficiencies associated with dystrophin-deficient respiratory and/or diaphragm deficiency.

The clinical course of DMD is characterized by the development of both spinal deformity and progressive respiratory insufficiency secondary to failure of the breathing apparatus. Without the muscular strength necessary to take regular deep breaths, the lungs and chest wall stiffen, limiting the ability to take in sufficient oxygen to meet metabolic requirements. Even in the earliest stages of the disease, when few if any respiratory symptoms have been reported, pulmonary functions begin to decline at fairly consistent, age-related rates.13 At ages 7-10, percent of predicted forced vital capacity (FVC) declines at an average annual rate of −0.3%; between ages 10-20, −8.5%; and after age 20, −6.2%. Finally, at mean age of 21.53, percent predicted FVC falls to under 10% and respiratory failure ensues. Because most subjects with DMD are confined to wheelchairs by the age of twelve, immobility contributes progressively to their airway clearance difficulties. The combined effects of immobility and ineffective cough contribute to the accumulation of pooled secretions, Pooled secretions provide fertile breeding ground for bacteria and contribute to a host of pulmonary complications. These complications include recurrent infection leading to tracheitis, bronchitis and pneumonia; impaired gas exchange; tissue damage; and bronchiectasis. In addition, such pooled secretions can contribute to ventilation-perfusion mismatch with consequent hypoxia, tachypnea and increased work of breathing.

In some embodiments, the present invention relates to a method for the treatment and/or prevention of a respiratory deficiency associated with a deficiency of the expiratory muscles and/or a deficiency of the diaphragm muscle in a subject. In various embodiments, the method comprises administering to the subject in need thereof, a therapeutically effective amount of a poloxamer as described herein, for example, a therapeutically effective dose of poloxamer P-188. In other embodiments, the present invention also relates to methods for increasing the tidal volume, tidal volume/body weight ratio, and decreasing diaphragm muscle fiber degeneration in a subject with a respiratory deficiency, the method comprising, administering to a subject in need of increasing the tidal volume, tidal volume/body weight ratio, and decreasing diaphragm muscle fiber degeneration, a therapeutically effective amount of a poloxamer. In various embodiments of the methods described herein, therapeutically effective amounts of the poloxamer are administered in pharmaceutical compositions comprising a therapeutically effective amount of a poloxamer and a pharmaceutically acceptable excipient. In one embodiment, the therapeutic doses for the methods for treating and/or preventing a respiratory deficiency can be administered intravenously, intraperitoneally, intramuscularly, subcutaneously, or combinations thereof to the subject in need thereof. In some embodiments, such parenteral administration methods can use therapeutically effective doses of a poloxamer in highly concentrated forms i.e. from about 25 mg to about 45,000 mg per 0.1-1,000 mL or less liquid volume, for example, 1,000 mL or less, 900 mL or less, or 800 mL or less, or 700 mL or less, or 600 mL or less, or 500 mL or less, or 250 mL or less, or 200 mL or less, or 150 mL or less, or 100 mL or less, or 75 mL or less, or 50 mL or less, or 25 mL or less, or 20 mL or less, or 10 mL or less, or 1 mL or less, administered at least once per day, for example, at least once per day, or at least twice per day, or every 4 hours, or every 8 hours, or every 12 hours, or every 24 hours, or every 36 hours, or every 48 hours, or one to five times per week, e.g. once per week, or once per two weeks, or once per month, or combinations thereof, for a period of 1-52 weeks, or 1-10 weeks, or at frequencies and/or amounts that improves overall therapy, reduces, avoids or eliminates symptoms, reduces side-effects or causes of a disease or disorder, or enhances the therapeutic efficacy of another therapeutic agent.

In some embodiments, parenteral administration of a pharmaceutical composition comprising a therapeutically effective amount of a poloxamer, (for example, any one or more of P138, P-188, P237, P288, P124, P338, and P407) to a subject with a respiratory deficiency associated with diaphragm muscular deficiency, for example, respiratory deficiencies associated with dystrophin-deficient diaphragm muscle can be accomplished by intravenous (for example, an infusion, or part of an infusion therapy) or subcutaneous injection. The pharmaceutical composition can be formulated in a volume of 0.1-1,000 mL with at least one pharmaceutically acceptable excipient, and administered to a subject in need thereof, at least once per day, or at least twice per day, or every 4 hours, or every 8 hours, or every 12 hours, or every 24 hours, or every 36 hours, or every 48 hours, or one to five times per week, e.g. once per week, or once per two weeks, or once per month, or combinations thereof, for a period of 1-52 weeks, or 1-10 weeks, or chronically for an indefinite period while symptoms persist, or at frequencies and/or amounts that improves overall therapy, reduces, avoids or eliminates one or more symptoms, reduces side-effects or causes of a disease or disorder, or enhances the therapeutic efficacy of another therapeutic agent. In some embodiments, the therapeutically effective amount to be dosed daily and/or weekly, and can be determined through appropriately controlled clinical trials, or through the use of a titration regimen of carefully determined therapeutically effective doses as described herein. In some embodiments, the subject in need thereof is dosed daily and/or weekly with a composition, pharmaceutical composition, or formulation comprising therapeutically effective amounts of a poloxamer ranging from about 0.01 mg/kg to about 500 mg/kg, preferably from about 0.1 mg/kg to about 300 mg/kg, or from about 0.1 mg/kg to about 30 mg/kg, or from about 0.1 mg/kg to about 3 mg/kg.

In various embodiments, methods for treating a subject with a respiratory deficiency associated with a diaphragm muscular deficiency comprises, administering to such subject, a therapeutically effective amount of a poloxamer such that after said administration of said pharmaceutical composition, the subject experiences an improvement in one or more respiratory deficiency criteria, selected from tidal volume, tidal volume/body weight ratio, and diaphragm muscle fiber degeneration. In some embodiments, an improvement in tidal volume of at least 1-12 percent, for example, an improvement of at least 1%, or at least 2%, or at least 3%, or at least 4%, or at least 5%, or at least 6%, or at least 7%, or at least 8%, or at least 9%, or at least 10%, or at least 11%, or at least 12%, is achieved when the subject having a respiratory deficiency associated with a diaphragm muscular deficiency is administered a composition, pharmaceutical composition, or formulation comprising a therapeutically effective amount of a poloxamer ranging from about 0.01 mg/kg to about 500 mg/kg, preferably from about 0.1 mg/kg to about 300 mg/kg, or from about 0.1 mg/kg to about 30 mg/kg, or from about 0.1 mg/kg to about 3 mg/kg. In some embodiments, the improvement in tidal volume capacity in a subject in need thereof, administered with a therapeutically effective amount of a poloxamer is from about 1% to about 20%, or from 3% to about 15%, or from about 6% to about 12 percent, or from about 9% to about 12 percent, or from 3% to about 15 percent, or from 5% to about 13% as compared to tidal volume capacity of the subject prior to commencement of the therapeutic treatment with the compositions, pharmaceutical compositions, or formulations comprising a poloxamer as described herein.

In some embodiments, an improvement in tidal volume/body weight ratio of at least 1-25 percent, for example, an improvement of at least 1%, or at least 2%, or at least 3%, or at least 4%, or at least 5%, or at least 6%, or at least 7%, or at least 8%, or at least 9%, or at least 10%, or at least 11%, or at least 12%, or at least 13%, or at least 14%, or at least 15%, or at least 16%, or at least 17%, or at least 18%, or at least 19%, is achieved when the subject having a respiratory deficiency is administered a composition, pharmaceutical composition, or formulation comprising therapeutically effective amounts of a poloxamer ranging from about 0.01 mg/kg to about 500 mg/kg, preferably from about 0.1 mg/kg to about 300 mg/kg, or from about 0.1 mg/kg to about 30 mg/kg, or from about 0.1 mg/kg to about 3 mg/kg. In some embodiments, the improvement in tidal volume/body weight ratio in a subject in need thereof administered with a therapeutically effective amount of a poloxamer is from about 1% to about 25%, or from 3% to about 20%, or from about 6% to about 20 percent, or from about 9% to about 20 percent, or from 10% to about 25 percent, or from 10% to about 20% as compared to the tidal volume/body weight ratio of the subject prior to commencement of the therapeutic treatment with the compositions, pharmaceutical compositions, or formulations comprising a poloxamer as described herein.

In some embodiments, an improvement in diaphragm muscle fiber degeneration, is shown as a reduction in the percentage of diaphragm muscle fibers with centralized nuclei after administration with a therapeutically effective amount of a poloxamer when compared to no treatment at all. In some embodiments, treatment of a subject in need thereof with a therapeutically effective amount of a poloxamer reduces the percentage of diaphragm muscle fibers with centralized nuclei sampled in the subject to less than 1-25 percent, for example, less than 25%, less than 23%, or less than 21%, or less than 19%, or less than 17%, or less than 15%, or less than 13%, or less than 11%, or less than 9%, or less than 7%, or less than 5%, or less than 3%, or less than 1%, when compared to the percentage of diaphragm muscle fibers with centralized nuclei sampled in the subject in the absence of a composition, pharmaceutical composition, or formulation comprising therapeutically effective amounts of a poloxamer.

In some of the above embodiments related to an improvement in one or more respiratory deficiency criteria, selected from tidal volume, tidal volume/body weight ratio, and diaphragm muscle fiber degeneration, the amount of poloxamer (for example, any one or more of poloxamers P138, P-188, P237, P288, P124, P338, and P407) administered to the subject in each dose and/or therapeutically effective amount ranges from about 0.01 mg/kg to about 500 mg/kg, preferably from about 0.1 mg/kg to about 300 mg/kg, or from about 0.1 mg/kg to about 30 mg/kg, or from about 0.1 mg/kg to about 3 mg/kg, or from about 5 mg to 45,000 mg, or from about 50 mg to about 40,000 mg, or from about 50 mg to about 30,000 mg, or from about 50 mg to about 20,000 mg, or from about 50 mg to about 10,000 mg, or from about 50 mg to about 5,000 mg, or from about 50 mg to about 4,000 mg, or from about 50 mg to about 3,000 mg, or from about 50 mg to about 2,000 mg, or from about 50 mg to about 1,500 mg, or from about 50 mg to about 1,000 mg, administered per day, or per week, to a subject in need thereof. In some embodiments, each dose and/or therapeutically effective amount is administered to a subject in need thereof, at least once per day, or at least twice per day, or every 4 hours, or every 8 hours, or every 12 hours, or every 24 hours, or every 36 hours, or every 48 hours, or one to five times per week, e.g. once per week, or once per two weeks, or once per month, or combinations thereof, for a period of 1-52 weeks, or 1-10 weeks, or administered indefinitely, or at frequencies and/or amounts that improves the overall therapy, reduces, avoids or eliminates symptoms, reduces side-effects or causes of a disease or disorder, or enhances the therapeutic efficacy of another therapeutic agent. In some embodiments, the poloxamer is P-188.

In some embodiments, an exemplary method comprises treating a subject with a respiratory deficiency (for example, a respiratory deficiency associated with an expiratory or a diaphragm muscular deficiency) by administering to such subject, a pharmaceutical composition comprising a therapeutically effective amount of poloxamer P-188 ranging from 0.1 mg/kg to about 500 mg/kg, (wt/wt %) such that after said administration of said pharmaceutical composition, the subject experiences an improvement in one or more respiratory deficiency criteria, selected from tidal volume, tidal volume/body weight ratio, and diaphragm muscle fiber degeneration. In some embodiments, the method provides administering the pharmaceutical composition to a subject in need thereof, comprising a therapeutically effective amount of a poloxamer P-188, at least once per day, or at least twice per day, or every 4 hours, or every 8 hours, or every 12 hours, or every 24 hours, or every 36 hours, or every 48 hours, or one to five times per week, e.g. once per week, or once per two weeks, or once per month, or combinations thereof, for a period of 1-52 weeks, or 1-10 weeks, or administered indefinitely, or at frequencies and/or amounts that improves the overall therapy, reduces, avoids or eliminates symptoms, reduces side-effects or causes of a disease or disorder, or enhances the therapeutic efficacy of another therapeutic agent.

In some of these embodiments, the therapeutically effective dose of poloxamer P-188 is an amount ranging from 0.1 mg/kg to about 500 mg/kg, (wt/wt %) (for example, from about 0.1 mg/kg to about 300 mg/kg, or from about 0.1 mg/kg to about 30 mg/kg, or from about 0.1 mg/kg to about 3 mg/kg, or from about 5 mg to 45,000 mg, or from about 50 mg to about 40,000 mg, or from about 50 mg to about 30,000 mg, or from about 50 mg to about 20,000 mg, or from about 50 mg to about 10,000 mg, or from about 50 mg to about 5,000 mg, or from about 50 mg to about 4,000 mg, or from about 50 mg to about 3,000 mg, or from about 50 mg to about 2,000 mg, or from about 50 mg to about 1,500 mg, or from about 50 mg to about 1,000 mg), which may be administered to the subject, one or more times per day, or one or more times per week, for a period of 1-52 weeks, or 1-10 weeks, or administered indefinitely, or at frequencies and/or amounts that improves the overall therapy, reduces, avoids or eliminates symptoms, reduces side-effects or causes of a disease or disorder, or enhances the therapeutic efficacy of another therapeutic agent to a subject in need thereof.

Methods for objective evaluation of the respiratory deficiency and the efficacy of treatment can include: oxyhemoglobin saturation by pulse oximetry, spirometric measurements of FVC, FEV1, and maximal mid-expiratory flow rate, maximum inspiratory and expiratory pressures, and peak cough flow. Awake carbon dioxide tension should be evaluated at least annually in conjunction with spirometry. Where available, capnography is ideal for this purpose. Arterial blood gas analysis is not necessary for routine follow-up of patients with DMD. If capnography is not available, then a venous or capillary blood sample should be obtained to assess for the presence of alveolar hypoventilation. Additional measures of pulmonary function and gas exchange may be useful, including lung volumes, assisted cough peak flow, and maximum insufflation capacity. Annual laboratory studies in patients requiring a wheelchair for ambulation should include a complete blood count, serum bicarbonate concentration, and a chest radiograph.

The use of respiratory evaluation techniques described herein can also be used to measure the therapeutic efficacy of the above recited methods and pharmaceutical compositions for the treatment of respiratory deficiencies associated with obstructive sleep apnea, oropharyngeal aspiration, gastroesophageal reflux, and asthma.

### EXAMPLES

### Example 1

### Dosing of P-188 to Treat Respiratory Dysfunction in the Mdx Mouse Model of Duchenne Muscular Dystrophy

Animals. Male C57BL/10SnJ, hereafter referred to as wild-type (WT), and male mdx C57B1/10ScSn-Dmdmdx mice were used in the study. All protocols were approved by the University of Michigan (UM) UCUCA and all studies were performed in facilities run by the University of Michigan Unit of Laboratory Animal Medicine. Mice were housed on a 12 hr dark-light cycle and provided water and chow ad libitum. Mice were purchased from Jackson Laboratories and aged in UM facilities. All ages given are ±2 weeks. Mice were randomized into groups upon receipt from Jackson Laboratories in order to minimize the need for additional cages. At 7 months of age, treatment with Poloxamer P-188 (P-188), saline or prednisone was initiated. All compounds were administered subcutaneously. Animals, dosed daily, received saline or P-188 at 300 mg/kg, 30 mg/kg or 3 mg/kg in a volume of 0.1 ml or prednisone 1 mg/kg (0.1 ml).

Respiration was monitored using a Buxco Whole Body Plethysmography (WBP) monitoring chambers for mouse Cat. No. PLY4211 (Buxco Research Systems, Wilmington, N.C.) according to the manufacturer's instructions. In this protocol animals are unrestrained and un-anesthetized. Monitoring was performed in the room in which the animals were housed. The mice were monitored for 2 months prior to initiation of treatment to acclimate the mice to the procedure and establish baseline respiratory parameters done with the last weeks reading prior to initiation of dosing. Mice were randomized into groups based on tidal volume measurements. Upon initiation of dosing, mice were monitored every 2 weeks. Mice were placed in monitoring chambers and allowed to acclimate for 15 minutes prior to recording respiratory parameters. Respiration was then monitored for 10 minutes. Each mouse was monitored in the same chamber for each reading throughout the study to minimize variability.

During acclimation and measurements, the room door was shut and technician movement and room noise was kept to a minimum to allow for complete acclimation. Longer acclimation time (30 minutes) did not affect the respiratory readings. Chambers were wiped clean between animals. Data was monitored and collected using FinePoint software (Cat. No. SFT4000) purchased from Buxco Research Systems (Wilmington, N.C.). This data was transferred to GraphPad PRIZM® software for graphing and statistical analysis of the data. Comparisons between groups of daily, every other day or weekly dosed animals were done by one-way ANOVA using the Neumann-Keuls post-hoc test. Significance was P<0.05. N=10-12 animals per group.

Abbreviations used include the following (units): F, respiration rate (breaths/minute), TV, tidal volume, (ml); TV/BWT, ratio of tidal volume to body weight, (mL/kg); MV, minute volume, (mL/min); Penh, enhanced pause, (no units); Rpef, the ratio of the time from start of expiration to peak expiratory flow to Te (no units); PIF, peak inspiratory flow, (mL sec); PEF, peak expiratory flow, (mL/sec); Te, expiratory time (sec); Ti, inspiratory time, (sec); Tr, relaxation time, (sec).

Respiratory function was diminished in mdx mice at 7 months of age. Respiration measurements were initiated in mdx and wild-type mice at 5 months of age and continued every 2 weeks for two months to acclimate the mice to the WBP chamber and establish baseline respiratory parameters. Baseline values (Table 1) shown were from the last measurement prior to dosing when the mice were 7 months of age±2 weeks. The mice were randomized into groups based on baseline TV values. At 7 months of age there were several significant differences in respiratory function between wild-type and mdx groups at baseline, which were similar to those results reported previously for mdx mice at 6 months of age. All of the mdx groups had a lower respiration rate than wild-type mice although some groups of mdx mice were not significantly lower. In addition, compared to the saline-treated wild-type (wild-type saline) group, the TV/BW ratio value was lower in all of the mdx groups although in the prednisone group this decrease did not reach significance (Table 1).

TABLE 1
Baseline measurements on wild type and mdx mouse groups at age 7 months^{g}.

| | Wild type | Wild type | | | | | Mdx |
|---|---|---|---|---|---|---|---|
| Parameter | Saline | 300 | Mdx Saline | Mdx 300 | Mdx 30 | Mdx 3 | Prednisone |
| F | 426 ± 27^{d} | 424 ± 26^{d} | 391 ± 19^{a} | 387 ± 32^{b} | 404 ± 28 | 414 ± 28 | 404 ± 16 |
| TV (mL) | 0.324 ± 0.024 | 0.328 ± 0.025 | 0.314 ± 0.017 | 0.320 ± 0.023 | 0.335 ± 0.023 | 0.323 ± 0.021 | 0.321 ± 0.019 |
| TV/BW | 9.3 ± 0.8^{e} | 8.8 ± 0.9 | 8.4 ± 0.5^{b} | 8.5 ± 0.7^{a} | 8.2 ± 0.6^{c} | 8.3 ± 0.5^{b} | 8.7 ± 0.5 |
| (mL/Kg) | | | | | | | |
| MV (mL/min) | 137 ± 10^{d} | 136 ± 11^{d} | 121 ± 10^{a} | 122 ± 13^{a} | 133 ± 12 | 132 ± 11 | 127 ± 8 |
| Penh | 0.427 ± 0.084 | 0.445 ± 0.051 | 0.424 ± 0.043 | 0.421 ± 0.041 | 0.503 ± 0.084^{ad} | 0.443 ± 0.057 | 0.470 ± 0.046 |
| Rpef | 0.404 ± 0.049^{d} | 0.375 ± 0.039 | 0.325 ± 0.048^{b} | 0.310 ± 0.071^{c} | 0.296 ± 0.058^{c} | 0.350 ± 0.060^{a} | 0.335 ± 0.027^{b} |
| PIF (mL/sec) | 10.89 ± 0.82 | 10.46 ± 0.82 | 10.18 ± 0.72 | 10.08 ± 0.95 | 10.79 ± 0.83 | 10.70 ± 0.73 | 10.27 ± 0.63 |
| PEF | 5.36 ± 0.49^{d} | 5.29 ± 0.33 | 4.80 ± 0.40^{d} | 4.89 ± 0.40 | 5.54 ± 0.55^{e} | 5.23 ± 0.46 | 5.10 ± 0.42 |
| Ti (sec) | 0.049 ± 0.003 | 0.051 ± 0.002 | 0.052 ± 0.002 | 0.053 ± 0.004^{b} | 0.052 ± 0.003 | 0.051 ± 0.003 | 0.053 ± 0.002^{a} |
| Te (sec) | 0.114 ± 0.014 | 0.111 ± 0.010^{d} | 0.128 ± 0.011 | 0.129 ± 0.017 | 0.121 ± 0.012 | 0.118 ± 0.013 | 0.123 ± 0.009 |
| Tr (sec) | 0.071 ± 0.011 | 0.067 ± 0.007 | 0.076 ± 0.008 | 0.078 ± 0.011 | 0.071 ± 0.007 | 0.071 ± 0.009 | 0.072 ± 0.006 |

^{a}P < 0.05 vs. wild type saline
^{b}P = 0.001-0.01 vs. wild type saline
^{c}P < 0.001 vs. wild type saline
^{d}P < 0.05 vs. mdx saline
^{e}P = 0.001-0.01 vs. mdx saline
f < 0.001 vs. mdx saline
^{g}all values are mean ± S.D.

Rpef, a parameter influenced by airway obstruction and residual activity of inspiratory muscles during the initiation of expiration, was lower in all mdx groups compared to wild-type groups. Other parameters are essentially similar between mdx and wild-type mice except for some sporadic differences between groups at baseline.

Chronic treatment with Poloxamer P-188 improves tidal volume in mdx mice.

Wild-type mice were dosed once-a-day (QD) subcutaneously (s.q.) with saline or 300 mg/kg of P-188 while mdx mice were dosed QD s.q. with saline, 3, 30 or 300 mg/kg of P-188 or 1 mg/kg of prednisone for 22 weeks. Respiration was monitored every 2 weeks by WBP. Table 2 shows measurements at 22 weeks of dosing once per day when mice were 1 year of age±2 weeks.

TABLE 2
Respiratory function in wild type and mdx mice after 22 weeks of treatment at 1 year of age^{g}

| | | | | | | | Mdx |
|---|---|---|---|---|---|---|---|
| Parameter | Control Saline | Control 300 | Mdx Saline | Mdx 300 | Mdx 30 | Mdx 3 | Prednisone |
| F | 332 ± 35 | 337 ± 24 | 326 ± 27 | 324 ± 32 | 308 ± 31 | 317 ± 43 | 328 ± 30 |
| (breaths/min) | | | | | | | |
| TV (mL) | 0.385 ± 0.035^{f} | 0.399 ± 0.029^{f} | 0.325 ± 0.019^{c} | 0.320 ± 0.033^{c} | 0.353 ± 0.024^{ad} | 0.364 ± 0.018^{a} | 0.332 ± 0.021^{c} |
| TV/BW | 11.0 ± 0.7^{f} | 11.1 ± 0.9^{f} | 8.8 ± 1.0^{c} | 8.8 ± 1.2^{c} | 9.8 ± 0.9^{ad} | 9.9 ± 0.5^{ad} | 9.9 ± 1.2^{ad} |
| (mL/kg) | | | | | | | |
| MV (ml/min) | 126 ± 15 | 131 ± 12^{f} | 105 ± 12^{b} | 100 ± 7^{c} | 106 ± 14^{b} | 112 ± 15^{a} | 107 ± 11^{b} |
| Penh | 0.334 ± 0.051 | 0.319 ± 0.042^{d} | 0.431 ± 0.024^{a} | 0.389 ± 0.071 | 0.453 ± 0.114^{b} | 0.389 ± 0.088 | 0.393 ± 0.080 |
| Rpef | 0.411 ± 0.093 | 0.485 ± 0.065 | 0.418 ± 0.080 | 0.480 ± 0.058 | 0.409 ± 0.107 | 0.469 ± 0.104 | 0.496 ± 0.085 |
| PIF (mL/sec) | 10.2 ± 0.9 | 10.7 ± 1.08 | 8.0 ± 0.94^{c} | 7.6 ± 0.80^{c} | 7.9 ± 1.2^{c} | 8.4 ± 1.2^{c} | 8.3 ± 0.8^{c} |
| PEF (mL/sec) | 4.8 ± 0.5 | 5.0 ± 0.4^{f} | 4.1 ± 0.5^{b} | 3.9 ± 0.3^{c} | 4.2 ± 0.5^{b} | 4.5 ± 0.5 | 4.2 ± 0.4^{b} |
| Ti (sec) | 0.063 ± 0.008 | 0.062 ± 0.006 | 0.069 ± 0.007 | 0.069 ± 0.067 | 0.073 ± 0.009^{a} | 0.072 ± 0.015 | 0.068 ± 0.006 |
| Te (sec) | 0.149 ± 0.021 | 0.149 ± 0.011 | 0.143 ± 0.015 | 0.145 ± 0.021 | 0.148 ± 0.017 | 0.147 ± 0.023 | 0.149 ± 0.022 |
| Tr (sec) | 0.097 ± 0.014 | 0.099 ± 0.010 | 0.086 ± 0.010 | 0.089 ± 0.015 | 0.087 ± 0.009 | 0.092 ± 0.016 | 0.094 ± 0.018 |

^{a}P < 0.05 vs. control saline
^{b}P = 0.001-0.01 vs. control saline
^{c}P < 0.001 vs control saline
^{d}P < 0.05 vs. mdx saline
^{e}P = 0.001-0.01 vs. mdx saline
^{f}<0.001 vs mdx saline
^{g}all values are mean ± S.D.

Significant differences between the wild-type saline and mdx saline groups and the treatment groups are indicated. At 22 weeks of dosing there were no significant differences between the wild-type groups treated with saline and 300 mg/kg of P-188 indicating no deleterious effect on respiration parameters at a high dose. F, Rpef, Ti, Te and Tr were similar in all groups in the study with some minor exceptions. MV was lower in all mdx groups compared to wild-type saline. PIF was significantly lower in all mdx groups compared to wild-type saline. PEF was significantly lower in the mdx groups compared to wild-type saline except for mdx mice dosed at 3 mg/kg of P-188. However PEF in the mdx 3 mg/kg group was not statistically different from the mdx saline group. Finally, the mdx group of mice treated with 300 mg/kg of P-188 showed no significant improvement in any respiratory parameter.

In contrast, TV was significantly higher in mdx mouse groups receiving 3 and 30 mg/kg of P-188 compared to mdx saline. TV was 9% and 12% higher in the 30 and 3 mg/kg P-188 treated groups, respectively. The TV/BW increase was 11% and 12.5% for the 30 and 3 mg/kg dose groups, respectively (Table 2). An increase in TV/BW was also seen in the prednisone treated mdx group compared with mdx saline at 22 weeks of treatment, however, this increase was due to a loss of body weight and not due to improvement in TV (Table 2). Table 3 shows a comparison of the baseline and 22-week treatment results for TV and TV/BW dosed once per day for each of the groups. It can be seen that both of the wild-type groups had significantly improved TV and TV/BW with age. A small increase in TV but not TV/BW was also noted in the mdx saline group over this same period. In the mdx groups treated with 30 and 3 mg/kg of P-188 there was a larger and highly significant 5% and 12.6% improvement in TV, respectively, and a 19.5% and 19.2% improvement in TV/BW after treatment with 30 and 3 mg/kg of P-188, respectively (Table 3).

TABLE 3
Effect of P-188 on respiratory parameters in wild type
and mdx mice after 22 weeks of daily dosing.¹

| Parameter | TV (ml) | | | TV/BW (ml/Kg) | | |
|---|---|---|---|---|---|---|
| Group | Baseline | 22 wks | P value^{a} | Baseline | 22 wks | P value^{a} |
| Wild-type | 0.324 ± 0.024 | 0.399 ± 0.029 | <0.0001 | 9.3 ± 0.8^{e} | 11.0 ± 0.7 | 0.0001 |
| Saline | | | | | | |
| Wild-type | 0.328 ± 0.025 | 0.399 ± 0.029 | <0.0001 | 8.8 ± 0.9 | 11.1 ± 0.9 | <0.0001 |
| 300 | | | | | | |
| mdx saline | 0.314 ± 0.017 | 0.325 ± 0.019 | 0.02 | 8.4 ± 0.5^{b} | 8.8 ± 1.0 | 0.3683 |
| mdx 300 | 0.320 ± 0.023 | 0.320 ± 0.033 | 0.94 | 8.5 ± 0.7^{a} | 8.8 ± 1.2 | 0.4627 |
| mdx 30 | 0.335 ± 0.023 | 0.353 ± 0.024 | 0.0017 | 8.2 ± 0.6^{c} | 9.8 ± 0.9 | <0.0001 |
| mdx 3 | 0.323 ± 0.021 | 0.364 ± 0.018 | 0.0007 | 8.3 ± 0.5^{b} | 9.9 ± 0.5 | <0.0001 |
| mdx | 0.321 ± 0.019 | 0.332 ± 0.021 | 0.104 | 8.7 ± 0.5 | 9.9 ± 1.2 | 0.0061 |
| prednisone | | | | | | |

¹data is mean ± S.D. N = 12 for all groups except mdx 3 mg/kg which was N = 11 at 22 wks.
²For F, all baseline vs. 22 wks, P < 0.0001, not significant (NS), not applicable (NA).
^{a}is the P value at 22 wks compared to baseline, paired t-test;

FIG. 1 shows TV measurements over the entire 22-week treatment period for the mdx groups treated with 30 or 3 mg/kg of P-188 or prednisone. During the early, intermediate and late stages of treatment with 30 mg/kg of P-188, there were weeks when a significant increase in TV, compared to mdx saline, was observed. This increase was observed in 6 of 11 readings. In contrast, the 3-mg/kg group showed improvement in TV only at the end of the treatment period. These results suggest the possibility that 30 mg/kg is more effective than the 3 mg/kg dose. WBP of unanesthetized, unrestrained mice has some inherent variability and this might account for the lack of a significant difference in some of the biweekly readings.

Diaphragm Histology

Analysis of centralized nuclei. One marker of degeneration is the amount of compensatory regeneration occurring within the muscle. Since newly regenerating fibers have centrally located nuclei, the percentage of fibers with centralized nuclei (CN) from the diaphragm of each animal was determined (FIG. 2A). Cells from the mdx saline group exhibited centralized nuclei in ˜25% of the fibers compared to ˜1% in the wild-type-saline group. P-188 treatment reduced the percentage of fibers containing CN in mdx mice significantly with the 30 mg/kg P-188 mdx group having the lowest percentage at 15%. This represents a 40% reduction in degeneration. There was no significant difference between any of the P-188 treated groups. The mdx prednisone group had nearly the same percentage of fibers with CN as the mdx saline group. These results suggest that subcutaneous administration of P-188 can slow the degeneration of diaphragm muscle cells in the diaphragm.

Analysis of fiber size and density. The cross-sectional area of regenerating or recently regenerated fibers is expected to be smaller than that of mature muscle fibers. The cross sectional area of approximately 50 muscle fibers was measured from each mouse (600 fibers/group). The results demonstrate that the fibers in all of the mdx groups were smaller than those in the wild-type groups (FIG. 2B) indicating the presence of recently regenerated fibers. The mdx saline group was different from all treatment groups in that its diaphragms had less fiber density and fibers were larger (FIG. 2C). The larger fiber size and lower density may indicate that muscle regeneration has decreased in 1-year old mdx mice. The mdx 300 group had a lower fiber density than the other P-188 treatment groups suggesting that the mdx 300 group may have other cell types within the muscle including fibroblasts and adipocytes or increased collagen deposition compared with the other treatment groups.

Analysis of fibrosis. Fibrosis was measured as the percentage of red area/total area in Masson's trichrome stained sections. The wild-type groups showed <1% staining while all of the mdx groups ranged between 39 and 48% fibrotic area. The difference between wild-type and mdx groups was highly significant (P<0.001) but there was no significant difference in fibrotic area between any of the mdx groups (data not shown). It is not clear why none of the mdx treatment groups had a lower fibrotic area than the saline treated mdx group, since a decrease in cardiac fibrosis was noted in P-188 treated dogs18 and prednisone has been shown to decrease diaphragm fibrosis in mdx mice. One hypothesis is that the fibrotic process was well underway at the age of 7 months (when dosing started) and that none of the treatments reversed the damage already done.

### DISCUSSION

Effects of P-188 on respiratory function and diaphragm structure. The studies reported here were undertaken to determine if P-188 treatment had beneficial effects on skeletal muscle and to test the hypothesis that P-188 acts on skeletal muscle that contracts regularly and frequently. The respiration study was a treatment initiated in mdx mice that were 7 months of age that had demonstrated respiratory deficits (Table 1). In this setting, P-188 at 30 mg/kg rapidly improved tidal volume early in the dosing regimen while the 3 mg/kg dose was effective after more prolonged dosing. The final effect of both doses was to improve TV compared to baseline readings and to improve TV compared to the mdx saline-treated group. This improvement was also maintained when body weight was taken into account. That the 30 mg/kg dose appeared to influence TV earlier than the 3 mg/kg dose suggests that it may be more effective than the 3 mg/kg dose. However, to establish that these doses fall in the dynamic region of the dose response curve, additional lower doses will be required. At the time that this study was conceived, only tidal volume and respiration rate were shown to be different between mdx and wild-type control mice. In this experimental example, the respiration rate was not greater in our mdx mice at 7 months of age and was not affected by P-188 treatment. However, respiration rate decrease over time in all groups possibly reflecting their acclimation to the routine associated with respiratory measurement.

There were other respiratory parameters that showed sporadic improvement in the P-188 and prednisone treated groups over the course of the dosing regimen. The variability was not surprising since the technique of WBP measures respiration in unrestrained conscious animals which introduces some variability into the measurements. While mice were given a 15-minute acclimation period prior to initiation of respiratory readings, not all mice exhibited the same level of activity in our studies. Similar results were obtained when the acclimation period was increased to 30 minutes.

Functional improvement was accompanied by changes in diaphragm histology that are consistent with the functional improvement (See FIG. 2). Damaged muscle fibers undergoing degeneration and regeneration are smaller than quiescent fibers (compare wild-type saline with mdx saline in FIG. 2B). As the number of fibers undergoing regeneration declines, muscle fiber cross-sectional area increases. Replacement of muscle fibers by fibroblasts and adipocytes becomes more prevalent as the regeneration process is exhausted. The diaphragm muscle from saline treated mdx mice had more centralized nuclei, larger fiber size and fewer muscle fibers per unit area than those from P-188 treated mdx mice. These results demonstrate that there is still active degeneration of muscle fibers in the saline treated mdx diaphragm muscles but that regeneration has slowed compared to P-188 and prednisone treatment, i.e. greater fiber cross-sectional area and less muscle fibers per unit area. In contrast, muscle fibers in the diaphragm of P-188 treated mdx mice showed less degenerating fibers (40% less centralized nuclei) and smaller fiber cross-sectional area suggesting that the regeneration has not been exhausted. This result is consistent with a cytoprotective effect of P-188 on the muscle fibers of the diaphragm. Prednisone treatment of mdx mice was expected to reduce inflammation associated with muscle degeneration and help to slow the loss of muscle fibers. Histological analysis of the diaphragm muscle from prednisone treated mdx mice shows a higher percentage of muscle fibers in the regeneration process than in the P-188 treated groups (See for example, FIG. 2, centralized nuclei). This suggests that prednisone does not protect myocytes as well as P-188. However, the cross-sectional areas in the prednisone treated mdx mouse diaphragms were similar to those in the P-188 treated mdx mice and smaller than those in the saline treated mdx mice suggesting that the regeneration process is still very active.

The histology results provide a mechanistic explanation for P-188's functional benefits. They indicate that regeneration process is active in the diaphragm of all mdx groups but less fibers appear to be involved in the P-188 treated groups. Such a protective effect might mean that at the time of examination diaphragm muscle from the low-dose P-188 treated mice have undergone fewer rounds of degeneration-regeneration and, as a result, have more regenerative capacity remaining.

It was surprising that the 300 mg/kg dose of P-188 had no effect on the respiratory parameters measured. To understand this result we performed a PK study. The data from the subcutaneous arm of this study indicated that plasma levels of P-188 were not dose proportional. At 300 mg/kg, the P-188 maximum plasma concentration and exposure were less than double that of the 30 mg/kg dose. More importantly the bioavailability of P-188 at the high dose was approximately 6-fold less that that at the lower dose (Table 4).

TABLE 4
Summary of the pharmacokinetics of P-188 dose intravenously and
subcutaneously to wild-type mice at different dose levels

| | Dose | | | | | | | | | |
|---|---|---|---|---|---|---|---|---|---|---|
| | level | t½ | Cmax | Tmax | AUClast | AUC∞ | Cl | Vss | Vz | |
| Dose Route | (mg/kg) | (h) | (μg/mL) | (h) | (μg * h/mL) | (μg * h/mL) | (mL/min/kg) | (mL/kg) | (mL/kg) | F (%) |
| Intravenous | 4.6 | 7.2 | NC | NC | 62 | 75 | 1.02 | 372 | 636 | NC |
| | 46 | 8.2 | NC | NC | 203 | 259 | 2.97 | 1435 | 2108 | NC |
| | 460 | 4.8 | NC | NC | 187 | 218 | 35.3 | 12078 | 14656 | NC |
| Subcutaneous | 30 | 3.9 | 70 | 2 | 369 | 423 | NC | NC | NC | 94.1 |
| | 100 | 2.3 | 133 | 2 | 674 | 699 | NC | NC | NC | 46.7 |
| | 300 | 3.7 | 118 | 1 | 639 | 708 | NC | NC | NC | 15.8 |

These results can be explained, in part, by the formation of P-188 micelles. The critical micelle concentration (CMC) is reported as 24-32 mg/mL at 37° C. and 105 mg/mL at 30° C. for P-188. The solution used to deliver 300 mg/kg of P-188 was 72.0 mg/mL in the PK study and 72 mg/mL in the efficacy study. Therefore, once injected, both solutions were above the critical micelle concentration of P-188 at 37° C. The influence of micelle formation on biological activity of P-188 has not been reported. However, based on our results, it appears that P-188 in micelles may be inactive. If micelle formation acts like a catalyst such that once initiated, more P-188 incorporates into the micelles so effectively the compound is lost to the system. Again, if large enough these micelles could behave like liposomes and be taken up by the reticuloendothelial system. This could result in a nearly complete loss of active P-188.

These results show the feasibility of using a once daily parenteral administration (for example, intravenous or subcutaneous administration) dosing regimen of P-188 for the treatment of respiratory deficits in subjects with DMD.

All patents mentioned in the above specification are herein incorporated by reference. Various modifications and variations of the described methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1. A method for treating or preventing a respiratory deficiency associated with an expiratory muscle deficiency or a diaphragm muscle deficiency in a subject, the method comprising administering to the subject in need thereof, a therapeutically effective amount of a poloxamer.

2. The method of claim 1, wherein said poloxamer is a purified or fractionated poloxamer, or an unfractionated poloxamer.

3. The method of claim 1, wherein said subject is a human subject.

4. The method of claim 1, wherein said poloxamer is administered via intravenous administration.

5. The method of claim 1, wherein said poloxamer is administered via subcutaneous administration.

6. The method of claim 1, wherein said subject is a dystrophin deficient subject.

7. The method of claim 6, wherein said subject has Duchenne muscular dystrophy.

8. The method of claim 1, wherein said subject has heart failure.

9. The method of claim 1, wherein said poloxamer is P-188.

10. The method of claim 1, wherein said therapeutically effective amount of said poloxamer is from about 0.01 mg per kilogram to about 500 mg per kilogram weight of said subject.

11. The method of claim 10, wherein said therapeutically effective amount of said poloxamer is from about 0.1 mg to 300 mg per kg; 0.1 mg to 200 mg per kg; 0.1 mg to 100 mg per kg; 0.1 mg to 50 mg per kg; or 0.1 mg to 30 mg per kg weight of said subject.

12. The method of claim 10, wherein said poloxamer is administered at a dosage level of about 0.1 mg to about 300 mg per kg weight of said subject.

13. A method for treating or preventing a respiratory deficiency associated with a diaphragm muscle deficiency in a subject, the method comprising administering to the subject in need thereof, a therapeutically effective amount of a poloxamer ranging from 5 mg to about 5,000 mg, of said subject dosed daily or weekly.

14. The method of claim 1, wherein the method comprises dosing said therapeutically effective amount of said poloxamer at least once per day, at least twice per day, every 4 hours, every 8 hours, every 12 hours, every 24 hours, every 36 hours, every 48 hours, one to five times per week, or combinations thereof.

15. The method of claim 1, wherein upon administration of said poloxamer to the subject, the subject experiences an improvement in one or more respiratory deficiency criteria, selected from tidal volume, tidal volume/body weight ratio, and diaphragm muscle fiber degeneration.

16. The method of claim 15, wherein the subject is administered a composition, pharmaceutical composition, or formulation comprising a therapeutically effective amount of a poloxamer ranging from about 0.1 mg/kg to about 300 mg/kg at least once per day, or at least twice per day, or every 4 hours, or every 8 hours, or every 12 hours, or every 24 hours, or every 36 hours, or every 48 hours, or one to five times per week, e.g. once per week, or once per two weeks, or once per month, or combinations thereof.

17. The method of claim 16, wherein said subject is a human subject.

18. The method of claim 16, wherein said poloxamer is administered via intravenous administration.

19. The method of claim 16, wherein said poloxamer is administered via subcutaneous administration.

20. The method of claim 16, wherein said subject is a dystrophin deficient subject.

21. The method of claim 20, wherein said subject has Duchenne muscular dystrophy.

22. The method of claim 16, wherein said subject has heart failure.

23. The method of claim 15, wherein said poloxamer is poloxamer P-188.

24. The method of claim 1, wherein said poloxamer is coadministered with one or more agents selected from the group consisting of streptomyocin, prednisone, deflazacort, azathioprine, cyclosporine, valproic acid, phenylbutyrate, sodium butyrate, M344, suberoylanilide hydroxamic acid, and PCT124.

25. The method of claim 1, wherein said poloxamer is selected from the group consisting of P138, P237, P288, P124, P338, and P407.

26. The method of claim 1, wherein a poloxamine and/or a polyglycidol is used instead of, or with, said poloxamer.
